SEQUENCE LISTING (37 sequences)

SEQ ID NO: 1 - protein, 274 aa, Artificial sequence
  Engineered T cell receptor (TCR) sequence
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 2 - protein, 274 aa, Artificial sequence
  Engineered T cell receptor (TCR) sequence
METLLGLLILWLQLQWVSSKQEVTQIPAALSVPEGENLVLNCSFTDSAIYNLQWFRQDPGKGLTSLLLVRPYQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 3 - protein, 274 aa, Artificial sequence
  Engineered T cell receptor (TCR) sequence
METLLGLLILWLQLQWVSSKQEVTQIPAALSVPEGENLVLNCSFTHSHIYNLQWFRQDPGKGLTSLLLIRSNQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 4 - protein, 274 aa, Artificial sequence
  Engineered T cell receptor (TCR) sequence
METLLGLLILWLQLQWVSSKQEVTQIPAALSVPEGENLVLNCSFTGSHIYNLQWFRQDPGKGLTSLLLIRSNQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 5 - protein, 274 aa, Artificial sequence
  Engineered T cell receptor (TCR) sequence
METLLGLLILWLQLQWVSSKQEVTQIPAALSVPEGENLVLNCSFTHSHIYNLQWFRQDPGKGLTSLLLIRSRQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - protein, 274 aa, Artificial sequence
  Engineered T cell receptor (TCR) sequence
METLLGLLILWLQLQWVSSKQEVTQIPAALSVPEGENLVLNCSFTGSHIYNLQWFRQDPGKGLTSLLLIRSEQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 7 - protein, 274 aa, Artificial sequence
  Engineered T cell receptor (TCR) sequence
METLLGLLILWLQLQWVSSKQEVTQIPAALSVPEGENLVLNCSFTGSHIYNLQWFRQDPGKGLTSLLLIRSDQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 8 - protein, 274 aa, Artificial sequence
  Engineered T cell receptor (TCR) sequence
METLLGLLILWLQLQWVSSKQEVTQIPAALSVPEGENLVLNCSFTNSHIYNLQWFRQDPGKGLTSLLLIRSDQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 9 - protein, 274 aa, Artificial sequence
  Engineered T cell receptor (TCR) sequence
METLLGLLILWLQLQWVSSKQEVTQIPAALSVPEGENLVLNCSFTGSSIYNLQWFRQDPGKGLTSLLLIRSSQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 10 - protein, 274 aa, Artificial sequence
  Engineered T cell receptor (TCR) sequence
METLLGLLILWLQLQWVSSKQEVTQIPAALSVPEGENLVLNCSFTKSEIYNLQWFRQDPGKGLTSLLLIRSSQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 11 - protein, 274 aa, Artificial sequence
  Engineered T cell receptor (TCR) sequence
METLLGLLILWLQLQWVSSKQEVTQIPAALSVPEGENLVLNCSFTKSNIYNLQWFRQDPGKGLTSLLLIRSDQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 12 - protein, 274 aa, Artificial sequence
  Engineered T cell receptor (TCR) sequence
METLLGLLILWLQLQWVSSKQEVTQIPAALSVPEGENLVLNCSFTNSHIYNLQWFRQDPGKGLTSLLLIQSHQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 13 - protein, 274 aa, Artificial sequence
  Engineered T cell receptor (TCR) sequence
METLLGLLILWLQLQWVSSKQEVTQIPAALSVPEGENLVLNCSFTHSHIYNLQWFRQDPGKGLTSLLLIHSHQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 14 - protein, 274 aa, Artificial sequence
  Engineered T cell receptor (TCR) sequence
METLLGLLILWLQLQWVSSKQEVTQIPAALSVPEGENLVLNCSFTSSGIYNLQWFRQDPGKGLTSLLLIRSDQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 15 - protein, 274 aa, Artificial sequence
  Engineered T cell receptor (TCR) sequence
METLLGLLILWLQLQWVSSKQEVTQIPAALSVPEGENLVLNCSFTNSGIYNLQWFRQDPGKGLTSLLLIRSSQREQTSGRLNASLDKSSGRSTLYIAASQPGDSATYLCAVRPGGAGSYQLTFGKGTKLSVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 16 - protein, 309 aa, Artificial sequence
  Engineered T cell receptor (TCR) sequence
MGSRLLCWVLLCLLGAGPVKAGVTQTPRYLIKTRGQQVTLSCSPISGHRSVSWYQQTPGQGLQFLFEYFSETQRNKGNFPGRFSGRQFSNSRSEMNVSTLELGDSALYLCASSPNMADEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSR

SEQ ID NO: 17 - protein, 274 aa, Artificial sequence
  Engineered T cell receptor (TCR) sequence
MLFSSLLCVFVAFSYSGSSVAQKVTQAQSSVSMPVRKAVTLNCLYETSWWSYYIFWYKQLPSKEMIFLIRQGSDEQNAKSGRYSVNFKKAAKSVALTISALQLEDSAKYFCALGEGGAQKLVFGQGTRLTINPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 311 aa, Artificial sequence
  Engineered T cell receptor (TCR) sequence
MSIGLLCCVAFSLLWASPVNAGVTQTPKFQVLKTGQSMTLQCAQDMNHNSMYWYRQDPGMGLRLIYYSASEGTTDKGEVPNGYNVSRLNKREFSLRLESAAPSQTSVYFCASRTRGGTLIEQYFGPGTRLTVTEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSR

SEQ ID NO: 19 - protein, 9 aa, Artificial sequence
  synthetic sequence
EVDPIGHLY

SEQ ID NO: 20 - protein, 9 aa, Artificial sequence
  synthetic sequence
ESDPIVAQY

SEQ ID NO: 21 - protein, 9 aa, Artificial sequence
  synthetic sequence
EVDPIGHVY

SEQ ID NO: 22 - protein, 9 aa, Artificial sequence
  synthetic sequence
ETDPVNHMV

SEQ ID NO: 23 - protein, 64 aa, Artificial sequence
  Engineered T cell receptor (TCR) sequence
SKQEVTQIPAALSVPEGENLVLNCSFTDSAIYNLQWFRQDPGKGLTSLLLIQSSQREQTSGRLN

SEQ ID NO: 24 - protein, 64 aa, Artificial sequence
  Engineered T cell receptor (TCR) sequence
SKQEVTQIPAALSVPEGENLVLNCSFTDSAIYNLQWFRQDPGKGLTSLLLVRPYQREQTSGRLN

SEQ ID NO: 25 - protein, 64 aa, Artificial sequence
  Engineered T cell receptor (TCR) sequence
SKQEVTQIPAALSVPEGENLVLNCSFTHSHIYNLQWFRQDPGKGLTSLLLIRSNQREQTSGRLN

SEQ ID NO: 26 - protein, 64 aa, Artificial sequence
  Engineered T cell receptor (TCR) sequence
SKQEVTQIPAALSVPEGENLVLNCSFTGSHIYNLQWFRQDPGKGLTSLLLIRSNQREQTSGRLN

SEQ ID NO: 27 - protein, 64 aa, Artificial sequence
  Engineered T cell receptor (TCR) sequence
SKQEVTQIPAALSVPEGENLVLNCSFTHSHIYNLQWFRQDPGKGLTSLLLIRSRQREQTSGRLN

SEQ ID NO: 28 - protein, 64 aa, Artificial sequence
  Engineered T cell receptor (TCR) sequence
SKQEVTQIPAALSVPEGENLVLNCSFTGSHIYNLQWFRQDPGKGLTSLLLIRSEQREQTSGRLN

SEQ ID NO: 29 - protein, 64 aa, Artificial sequence
  Engineered T cell receptor (TCR) sequence
SKQEVTQIPAALSVPEGENLVLNCSFTGSHIYNLQWFRQDPGKGLTSLLLIRSDQREQTSGRLN

SEQ ID NO: 30 - protein, 64 aa, Artificial sequence
  Engineered T cell receptor (TCR) sequence
SKQEVTQIPAALSVPEGENLVLNCSFTNSHIYNLQWFRQDPGKGLTSLLLIRSDQREQTSGRLN

SEQ ID NO: 31 - protein, 64 aa, Artificial sequence
  Engineered T cell receptor (TCR) sequence
SKQEVTQIPAALSVPEGENLVLNCSFTGSSIYNLQWFRQDPGKGLTSLLLIRSSQREQTSGRLN

SEQ ID NO: 32 - protein, 64 aa, Artificial sequence
  Engineered T cell receptor (TCR) sequence
SKQEVTQIPAALSVPEGENLVLNCSFTKSEIYNLQWFRQDPGKGLTSLLLIRSSQREQTSGRLN

SEQ ID NO: 33 - protein, 64 aa, Artificial sequence
  Engineered T cell receptor (TCR) sequence
SKQEVTQIPAALSVPEGENLVLNCSFTKSNIYNLQWFRQDPGKGLTSLLLIRSDQREQTSGRLN

SEQ ID NO: 34 - protein, 64 aa, Artificial sequence
  Engineered T cell receptor (TCR) sequence
SKQEVTQIPAALSVPEGENLVLNCSFTNSHIYNLQWFRQDPGKGLTSLLLIQSHQREQTSGRLN

SEQ ID NO: 35 - protein, 64 aa, Artificial sequence
  Engineered T cell receptor (TCR) sequence
SKQEVTQIPAALSVPEGENLVLNCSFTHSHIYNLQWFRQDPGKGLTSLLLIHSHQREQTSGRLN

SEQ ID NO: 36 - protein, 64 aa, Artificial sequence
  Engineered T cell receptor (TCR) sequence
SKQEVTQIPAALSVPEGENLVLNCSFTSSGIYNLQWFRQDPGKGLTSLLLIRSDQREQTSGRLN

SEQ ID NO: 37 - protein, 64 aa, Artificial sequence
  Engineered T cell receptor (TCR) sequence
SKQEVTQIPAALSVPEGENLVLNCSFTNSGIYNLQWFRQDPGKGLTSLLLIRSSQREQTSGRLN

(19) United States
(12) Patent Application Publication
(10) Pub. No.: US 2024/0299546 A1
(43) Pub. Date: Sep. 12, 2024

(54) HIGH POTENCY T CELL RECEPTORS FOR IMMUNOTHERAPY

(71) Applicant: The Board of Trustees of the Leland Stanford Junior University, Stanford, CA (US)

(72) Inventors: Kenan Christopher Garcia, Menlo Park, CA (US); Xiang Zhao, Redwood City, CA (US)

(21) Appl. No.: 18/279,191

(22) PCT Filed: Mar. 4, 2022
(86) PCT No.: PCT/US2022/018975

§ 371 Date: Aug. 28, 2023

Related U.S. Application Data
(60) Provisional application No. 63/158,131, filed on Mar. 8, 2021.

(51) Int. Cl. A61K 39/00; C07K 14/725; C12N 5/0783; C12N 15/10; C12N 15/86

(52) U.S. Cl. CPC A61K 39/464486; A61K 39/4611; A61K 39/4632; C07K 14/7051; C12N 5/0636; C12N 15/1037; C12N 15/86; C12N 2740/15043

(57) ABSTRACT

Engineered T cell receptor (TCR) sequences, cells expressing such sequences and methods of use thereof are provided. The engineered receptors are mutagenized in vitro, and selected for target activation potency in combination with selection for a pMHC affinity that is sufficiently low to reduce off-target cross-reactivity. In some embodiments the engineered TCR recognizes the tumor associated antigen (TAA), human MAGE-A3

### CROSS REFERENCE TO RELATED APPLICATION

The present application claims the benefit of and priority to U.S. Provisional Patent Application No. 63/158,131, filed Mar. 8, 2021, the entire disclosure of which is hereby incorporated by reference in its entirety.

### STATEMENT REGARDING FEDERALLY SPONSORED RESEARCH

This invention was made with Government support under contract 5R01AI103867 awarded by the National Institutes of Health. The Government has certain rights in the invention.

### INCORPORATION BY REFERENCE OF SEQUENCE LISTING PROVIDED AS A TEXT FILE

A Squence Listing is provided herewithin in a text file, (STAN-1832WO_SEQ_LIST_ST25.txt), created on Mar. 1, 2022, and having a size of 56,000 bytes. The contents of the text file are incorporated herein by reference in its entirety.

### BACKGROUND

Adaptive immune responses are initiated by T cell receptor (TCR) recognition of peptides presented by major histocompatibility molecules (pMHC). TCR engagement with an agonist pMHC leads to phosphorylation of CD3 immunoreceptor tyrosine-based activation motifs (ITAMs), initiating a cascade of downstream signaling that results in T cell activation. Despite the low 3D affinity (K_{D} 1-100 μM) of the TCR-pMHC interaction, the potency of the T cell response has been shown to generally reflect the strength, or duration, of a particular interaction.

TCR signaling is influenced by parameters other than the affinity of TCR for pMHC. For example, force-dependent interactions are a characteristic of agonist pMHC ligands. TCRs form catch bonds with agonist ligands, during which the bond lifetime of the interaction extends under force. Catch bonds represent a net gain of molecular interactions under force, revealing an additional level of dynamic diversity built-in as a proofreading mechanism to link TCR recognition and subsequent activation. This provides a triggering mechanism by which TCR ligation and activation can be coupled or decoupled to regulate TCR ligand discrimination.

Adoptive transfer of T lymphocytes with engineered specificity for tumor antigens is a promising approach to target cancer, with potent antitumor activity in patients receiving such treatment. However, because most tumor antigens are derived from self-proteins, it is difficult to isolate native high-affinity tumor specific T cells, and receptor sequences must be enhanced by ex vivo engineering.

Importantly, however, while considerable increases in TCR antigen affinity have been reported, even down to picomolar range, this level of affinity can increase the risk of treatment-induced toxicity. In some instances toxicity has been associated with “on target” reactivity, where the target antigen is expressed in normal cells, e.g. melanocytes expressing melanoma antigens.

Affinity maturation also increases the likelihood that the TCR will cross-react to other peptide-MHC molecules on tissues outside of cancer cells, leading to off target toxicity and possibly patient adverse events or death. This has been demonstrated with affinity-matured TCR that target the human tumor antigen MAGE-A3; these TCR-T cells crossreacted with a cardiac peptide called Titin with deadly results to patients.

This problem is an innate limitation of all TCR-T therapies because TCRs usually have low affinity and will not kill cells expressing self-antigens like those expressed on tumors. The present disclosure provides methods of screening, and useful TCR sequences, that address this issue.

### SUMMARY

Engineered T cell receptor (TCR) sequences, cells expressing such sequences and methods of use thereof are provided. The engineered receptors are mutagenized in vitro, and selected for target activation potency, in combination with selection for a pMHC affinity that is sufficiently low to reduce off-target cross-reactivity. The pMHC affinity is contained within an appropriate window; above this threshold level, efficacy and specificity are compromised. In some embodiments, cells expressing the engineered TCR are used for adoptive T cell therapy to treat cancer. In some embodiments the engineered TCR recognizes the tumor associated antigen (TAA): human MAGE-A3. In some embodiments the TAA is recognized in the context of human HLA-A1.

In some embodiments the engineered TCR specific for MAGE-A3 comprises an alpha chain (TCRα) of SEQ ID NO:1 or a mature version thereof lacking the signal sequence, and comprises at least one amino acid modification to enhance target activation potency, wherein the modification is made at one or more residues selected from D28, A30, I51, Q52, S53 and S54 (numbering relative to the mature protein sequence). In some embodiments the amino acid modification is an amino acid substitution. In some embodiments the amino acid substitution is selected from D28H/N/G/K/S; A30H/S/E/N/G; I51V; Q52R/H; S53P; S54Y/N/R/E/D/H. In some embodiments the TCRα has a sequence selected from SEQ ID NO:2-SEQ ID NO:15, or a variant derived therefrom. Variants may comprise at least about 90% sequence identity, at least 95% sequence identity, at least about 97%, sequence identity, at least about 99% sequence identity to a reference sequence of SEQ ID NO:2-15. In any of such embodiments the beta chain (TCRβ) may have the sequence set forth in SEQ ID NO:16 or a mature version thereof, lacking the signal sequence. The MAGE-A3 engineered TCR does not have significant affinity for human titin sequences.

An engineered TCR, e.g. a TCR specific for MAGE-A3, may have a 3D log K_{D} (μM) of from about 0.5 to about 100 μM, and may be from about 1 to about 100 μM, from about 1 to about 50 μM. The engineered TCR is desirably selected for target activation potency, as measured by any convenient assay, including without limitation T cell proliferation in response to antigen, release of IL-2 in response to antigen, upregulation of CD69 on a T cell in response to antigen, and the like.

In some embodiments the engineered TCR is specific for an HIV peptide presented by HLA-B35, based on amino acid modifications of TCR55 alpha chain (SEQ ID NO:17) and TCR55 beta chain (SEQ ID NO:18). The amino acid modifications include, without limitation, SEQ ID NO:17 A98D, A98E, A98F, A98Q, A98Y, A98H to make TCR55 activated by B35-HIV. Amino acid modification in TCR55 beta chain (SEQ ID NO:18) include, without limitation, A50D, A50E, A50F, A50H, A50N, A50Q, A50S, A50T, A50Y to make TCR55 activated by B35-HIV.

In some embodiments, an engineered cell is provided, which the cell has been modified by introduction of a engineered TCR coding sequence, usually modified by introduction of both a TCRα and TCRβ sequence. Any cell can be used for this purpose. In some embodiments the cell is a T cell, including without limitation naïve CD8⁺ T cells, cytotoxic CD8⁺ T cells, naïve CD4⁺ T cells, helper T cells, e.g. T_{H}1, T_{H}2, T_{H}9, T_{H}1, T_{H}22, T_{FH}; regulatory T cells, e.g. T_{R}1, natural T_{Reg}, inducible T_{Reg}; memory T cells, e.g. central memory T cells, effector memory T cells, NKT cells, γδ T cells and engineered variants of such T-cells including CAR-T cells; etc. In other embodiments the engineered cell is a stem cell, e.g. a hematopoietic stem cell, a lymphoid progenitor cell, etc. In some embodiments the cell is genetically modified in an ex vivo procedure, prior to transfer into a subject. The engineered cell can be provided in a unit dose for therapy, and can be allogeneic, autologous, etc. with respect to an intended recipient. Introduction of the coding sequence can be performed in vivo or in vitro, using any appropriate vector, e.g., viral vectors, integrating vectors, and the like. In some embodiments a gene editing system, including without limitation CRISPR-Cas9, is used to integrate the sequences into the genome of the engineered cell.

In some embodiments, a vector comprising a polynucleotide sequence encoding an engineered TCR sequence as described herein is provided, where the coding sequence may be operably linked to a promoter active in the desired cell. In some embodiments, the promoter may be constitutive or inducible. Various vectors are known in the art and can be used for this purpose, e.g. viral vectors, plasmid vectors, minicircle vectors, etc. which vectors can be integrated into the target cell genome, or can be episomally maintained. The vector may be provided in a kit.

In some embodiments a therapeutic method is provided, the method comprising introducing into a recipient in need thereof an effective dose of an engineered cell population, wherein the cell population has been modified by introduction of a sequence encoding an engineered TCR as disclosed herein. The cell population may be engineered ex vivo, and is usually autologous or allogeneic with respect to the recipient. The recipient may be treated for cancer by administration of the engineered cell population. The recipient may be treated with the engineered cell population in combination with additional therapeutic compositions or modalities, including immunotherapy, chemotherapy, radiation therapy, surgery, and the like as known in the art. The introduced T cells may increase killing of targeted cells expressing the cognate antigen.

In some embodiments methods are provided for selecting variants of a TCR, e.g. TCRα or TCRβ, for target activation potency in combination with selection for a pMHC affinity that is sufficiently low to reduce off-target cross-reactivity, which approach may be referred to as “catch bond fishing”. The screening is based on the finding that activation potency can be decoupled from binding affinity. By this method the pMHC affinity is selected so as to be contained within an appropriate window to reduce off-target toxicities.

The starting TCR for optimization may be a TCR specific for a target of interest, including without limitation known sequences to known targets. Antigens of interest include, without limitation, tumor associated antigens, including for example HER2, PSA, TRP-2, EpCAM, GPC3, mesothelin (MSLN), CEA, MUC1, MAGE, EGFR, etc., presented in a patient relevant MHC context, e.g. human HLA antigens. Also of interest pathogen antigens, e.g. viral antigens, bacterial antigens, and the like.

In such screening methods, a library is generated comprising amino acid variations at pre-determined amino acid residues on the TCR sequence for optimization. The residues selected for mutagenesis are usually within one or more of the CDR regions of the TCR. A TCRα sequence may be mutagenized and paired with a non-mutagenized TCRβ, or TCRβ sequence may be mutagenized and paired with a non-mutagenized TCRα. The library is introduced into mammalian cells for expression, including mammalian T cell lines. The cells are first selected for low affinity binding to the cognate pMHC, e.g. by binding to labeled pMHC tetramers, multimers, etc., and sorting by flow cytometry, etc., for low affinity binding, e.g. binding at a 3D log K_{D} (μM) of from about 0.1 to about 100 μM.

The low affinity TCR sequences are screened for the ability to activate T cells in response to antigen. Where the initial screening was done in T cells, the T cells may be directly screened; alternatively the sequences of low affinity binding TCR are introduced into T cells for activation screening. The population of T cells expressing TCRs with low antigen affinity are incubated with an antigen source, e.g. target cells expressing the cognate antigen, a pMHC substrate, antigen-presenting cells pulsed with antigenic peptide, etc., for a period of time sufficient to activate the T cells. The T cells are selected for high levels of activation, e.g. by proliferation, IL-2 release, CD69 upregulation, etc. Conveniently, upregulation of CD69 is selected by antibody staining and flow cytometry. Such selection may be based on relative values, where the cells in the top 20%, top 10%, top 5%, top 1% are selected. The resulting engineered TCR may be validated for low off-target cross-reactivity and high on-target activation.

In some embodiments kits are provided for screening, which may comprise, for example, cell lines suitable for screening, vectors for expression of the mutagenized TCR, pMHC tetramers for labeling cells, anti-CD69 antibodies for labeling cells, and the like.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Working flow of catch bond engineering of TCR. TCR libraries were synthesized as dsDNA with randomized residues. The library was cloned into lentiviral vector by Gibson assembly. The library of recombinant lentiviral vectors were used to produce the library of lentivirus to infect SKW-3 T cell line. The display of TCR library on SKW-3 T cells were detected by anti-TCR (clone IP26) staining. The T cell library was cocultured with antigen-presenting cells pulsed with 10 μM antigenic peptide for 14 hours, and the T cell library was stained with anti-CD69-APC and specific pMHC tetramer. Any clones with high-level anti-CD69 staining and low-level tetramer staining were sorted for further rounds of sorting or analysis.

FIG. 2. Sorting strategy of TCR catch bond engineering. In the round 0, the T cell library or WT TCR transfectant was stained with anti-CD69-APC and specific pMHC tetramer. The T cell library clones which have similar level of anti-CD69 and tetramer staining compared to WT TCR transfectants were sored to remove any high-affinity or auto-responsive clones. Afterwards, the T cell library was cocultured with antigen-presenting cells pulsed with 10 μM antigenic peptide for 14 hours, and the T cell library was stained with anti-CD69-APC and specific pMHC tetramer. Any clones with high-level anti-CD69 staining and low-level tetramer staining were sorted. The same sorting procedure as round 2 were repeated for 2-3 more rounds to further enrich certain mutants.

FIGS. 3A-3C. Design of TCR55 libraries. Based on the structure of B35-HIV-TCR55 (PDB ID: 6BJ3), residues on TCR55 alpha chain (Ser28, Lys69, Ala98) were selected and randomized into VRW codon as 55a library (A); residues on TCR55b beta chain CDR1 and CDR2 (Asn28, Ser31, Ala50 and Ser51) were selected and randomized into VRW codon as 55b12 library (B); residues on TCR55b beta chain CDR3 (Lys71, Thr95 and Leu100) were selected and randomized into VRW codon as 55b3 library) (C).

FIG. 4. 5 rounds of selection of TCR55 libraries. In each round, the T cell library was cocultured with KG-1 cells pulsed with 10 μM HIV peptide for 14 hours, and the T cell library was stained with anti-CD69-APC and HLA-B35-HIV tetramer. Any clones with high-level anti-CD69 staining and low-level tetramer staining were sorted. Gating is based on the anti-CD69 and B35-HIV tetramer staining of TCR55 WT transfectants.

FIGS. 5A-5D. TCR55a-A98H is a catch bond-engineered TCR which can be activated by B35-HIV. A. TCR55 WT, TCR55a-A98H, TCR55a-S28G or TCR55a-S28G A98H T cell transfectants were cocultured with KG-1 cells pulsed with titrated HIV peptide for 14 hours and stained with anti-CD69. The experiment was analyzed by flow cytometry. B-C. Surface plasmon resonance (SPR) experiment to measure the 3D binding affinity between immobilized B35-HIV and flowed TCR55a-A98H protein. D. Biomembrane force probe (BFP) experiment to measure the bond lifetime between B35-HIV protein and TCR55 WT or TCR55a-A98H T cell transfectants.

FIGS. 6A-6E. TCR55a-Ala98 is a hot spot for catch bond engineering. A. TCR55a-A98 mutation to D, E, F, Q, Y and H were made as T cell transfectants and stimulated by KG-1 cells pulsed with titrated HIV peptide for 14 hours and stained with anti-CD69. The experiment was analyzed by flow cytometry. B. TCR55a-A98 mutation to C, K, N, R, S, T and W were made as T cell transfectants and stimulated by KG-1 cells pulsed with titrated HIV peptide. Analysis was the same as A. C. The correlation between Emax and 3D binding affinity (K_{D}) of stimulatory TCR55a-A98 mutants. D. The correlation between EC₅₀ and 3D K_{D} of stimulatory TCR55a-A98 mutants. E. BFP measurement of bond lifetime of TCR55a-A98H, TCR55a-A98E and TCR55a-A98Q T cell transfectants interacting with B35-HIV protein.

FIGS. 7A-7B. Design of MAGE libraries. Based on the structure of HLA-A1-MAGEA3-MAG-IC3 (PDB ID: 5BRZ), residues on TCR alpha chain (Asp28, Ala30, Ser54 and Gln52) were selected and randomized into VRW codon as a library (A); residues on TCR beta chain (Thr54, Met98 and Asp100) were selected and randomized into VRW codon as b library (B).

FIG. 8. 3 rounds of selection of MAGE libraries. In each round, the T cell library was cocultured with antigen-presenting cells pulsed with 10 μM MAGEA3 peptide for 14 hours, and the T cell library was stained with anti-CD69-APC and HLA-A1-MAGEA3 tetramer. Any clones with high-level anti-CD69 staining and low-level tetramer staining were sorted. Gating is based on the anti-CD69 and HLA-A1-MAGEA3 tetramer staining of MAGEA3 WT TCR transfectants.

FIGS. 9A-9C. Multiple TCR mutants were identified to be activated by MAGEA3 tumor antigen. A. 8 high-potency mutants T cells were cocultured with 293T-HLA-A1 cells pulsed with titrated MAGEA3 peptide for 14 hours. T cells were stained with anti-CD69 and analyzed on flow cytometry. B. 5 intermediate-potency mutants T cells were cocultured with 293T-HLA-A1 cells pulsed with titrated MAGEA3 peptide for 14 hours. T cells were stained with anti-CD69 and analyzed on flow cytometry. C. 8 high-potency mutants T cells were cocultured with 293T-HLA-A1 cells pulsed with titrated TITIN peptide for 14 hours. T cells were stained with anti-CD69 and analyzed on flow cytometry.

FIGS. 10A-10C. Identification of several MAGE TCR mutants with high potency but lower affinity compared to A3A TCR. A. Correlation between Emax and HLA-A1-MAGEA3 tetramer stained-positive percentage of WT TCR, A3A TCR, 8 high-potency mutants and 5 intermediate-potency mutants. B. Correlation between Emax and 3D affinity (3D K_{D}) of immobilized HLA-A1-MAGEA3 binding to WT, A3A or 6 other selected TCR mutants. C. Correlation between EC50 and 3D K_{D} of immobilized HLA-A1-MAGEA3 binding to WT, A3A or 6 other selected TCR mutants.

FIG. 11. Toxicity screening. Repeat 1: human primary T cells cytotoxicity assay. Antigen-presenting cells: tumor cell lines (A375, HCT-116)-HLA-A1-MAGEA3+, 27a-5: one MAGE TCR mutant.

FIG. 12. Amino acid substitutions of all MAGE TCR mutants. All the mutants only have mutations in TCR alpha chain residues of Asp28, Ala30, Ile51, Gln52, Ser53 and Ser54. The specific mutated residues of each mutate are listed here.

FIG. 13. Alignment of a selected portion of the engineered MAGE TCR sequences.

FIGS. 14A-14P. Cytotoxicity and specificity of catch bond engineered MAGE-A3-specific TCR. (A-B) Killing of A375 melanoma cell line by different MAGE-A3-specific TCR transduced human primary T cells. (C-E) IFN-ψ, TNF, and cytotoxic granule release (CD107a staining) by different MAGE-A3-specific TCR transduced human primary T cells, induced by the A375 melanoma cell line. (F-G) Killing of HCT-116 colon cancer cell line by different MAGE-A3-specific TCR transduced human primary T cells. (H-J) IFN-ψ, TNF, and cytotoxic granule release (CD107a staining) by different MAGE-A3-specific TCR transduced human primary T cells, induced by the HCT-116 colon cancer cell line. (K-M) Cytotoxic granule release (CD107a staining), TNF, and IFN-γ by different MAGE-A3-specific TCR transduced human primary T cells, induced by HLA-A1+293T cells pulsed with a titration of MAGE-3 peptide. (N-P) Cytotoxic granule release (CD107a staining), TNF, and IFN-γ by different MAGE-A3-specific TCR transduced human primary T cells, induced by HLA-A1⁺ 293T cells pulsed with a titration of TITIN peptide. (A-P) Data are representative of 3 independent experiments. Data are shown as mean±SD of technical duplicates. ns: not significant; *: P<0.05; **: P<0.01; ***: P<0.001; ****: P<0.0001

FIGS. 15A-15E. Cross-reactivity screening of MAGE-A3 TCR variants by yeast-display pMHC library. A. Design of the single-chain HLA-A*01 yeast-display peptide library. The DNA peptide library design shows an NNK codon library for all positions except anchor positions P3 (GAK) and P9 (TAY) to maximize peptides displayed by HLA-A*01. The single-chain trimer construct is N-terminal to the Myc tag fused to Aga2 for expression on yeast. B. Increasing myc tag expression on yeast over rounds of selection represents enrichment of peptide HLA-A*01 and positive selection of the library. C. Heat map of round 4 selected peptides showing peptide position by amino acid accounting for the number of reads detected per peptide. Boxed amino acids represent the MAGE-A3 peptide (SEQ ID NO:19) EVDPIGHLY. Dark represents a more enriched amino acid in that position. D. MAGE-A3, TITIN, DMSO (black dot) and 60 predicted peptides (MAGE-A6; FAT2) were used to pulse 293T-HLA-A1 cells to stimulate SKW3 T cells expressing different TCRs for 14 hours. Peptides were MAGE-A3 (SEQ ID NO:19) EVDPIGHLY, TITIN (SEQ ID NO:20) ESDPIVAQY; MAGE-A6 (SEQ ID NO:21) EVDPIGHVY; FAT2 (SEQ ID NO:22) ETDPVNHMV. D. Anti-CD69-APC staining was performed and analyzed on flow cytometry. 293-HLA-A1 cells were pulsed with titrated MAGE-A3 (SEQ ID NO:19), TITIN (SEQ ID NO:20), MAGE-A6 (SEQ ID NO:21) or FAT2 (SEQ ID NO:22) peptides to stimulate SKW3 T cells expressing MAGE-A3 TCR variants for 14 hours. Anti-CD69-APC staining was performed and analyzed on flow cytometry.

FIGS. 16A-16S. Killing, cytokine responses, and granule release mediated by other MAGE-A3-specific TCR mutants. A. A1-MAGE-A3 tetramer staining and anti-CD69 staining of MAGE-A3 WT TCR SKW3 transfectants in each round of selection of the library. B. The correlation between Emax and percentage of HLA-A1-MAGE-A3 tetramer staining-high population of different MAGE-A3-specific TCR mutants in SKW3 cells. C. The correlation between log 10EC50 and 3D binding affinity K_{D} of selected MAGE-A3-specific TCR mutants binding to HLA-A1-MAGE-A3. (D-E) Killing of A375 melanoma cell line by different MAGE-A3-specific TCR transduced human primary T cells. (F-H) IFN-γ, TNF, and cytotoxic granule release (CD107a staining) by different MAGE-A3-specific TCR transduced human primary T cells stimulated by the A375 melanoma cell line. (I-J) Killing of HCT-116 colon cancer cell line by different MAGE-A3-specific TCR transduced human primary T cells. (K-M) IFN-γ, TNF, and cytotoxic granule release (CD107a staining) by different MAGE-A3-specific TCR transduced human primary T cells, stimulated by the HCT-116 colon cancer cell line. (N-P) Cytotoxic granule release (CD107a staining), TNF, and IFN-γ by different MAGE-A3-specific TCR transduced human primary T cells, stimulated by HLA-A1+ 293T cells pulsed with titrated MAGE-A3 peptide. (Q-S) Cytotoxic granule release (CD107a staining), TNF, and IFN-γ by different MAGE-A3-specific TCR transduced human primary T cells, stimulated by HLA-A1+293T cells pulsed with titrated TITIN peptide. (D-S) Data are representative of 3 independent experiments. Data are shown as mean±SD of technical duplicates. ns: not significant; *: P<0.05; **: P<0.01; ***: P<0.001; ****: P<0.0001

FIGS. 17A-17B. SPR experiments of MAGE-A3-specific TCR mutants binding to HLA-A1-MAGE-A3. A. SPR experiments of MAGE-A3-specific TCR mutants protein binding to HLA-A1-MAGE-A3. Biotinylated HLA-A1-MAGE-A3 monomer was immobilized on the streptavidin chip and the MAGE-A3-specific TCR mutant proteins were flowed through the chip. Determination of 3D affinity between MAGE-A3-specific TCR mutants and HLA-A1-MAGE-A3 by SPR. B. Equilibrium curves of MAGE-A3-specific TCR mutants binding to HLA-A1-MAGE-A3 pMHC at 25° C. Data shown was measured at equilibrium (black dots). Black lines show the fit to a 1:1 binding curve.

FIGS. 18A-18B. SPR experiments of MAGE-A3-specific TCR mutants binding to HLA-A1-TITIN. A. SPR experiments of MAGE-A3-specific TCR mutants protein binding to HLA-A1-TITIN. Biotinylated HLA-A1-TITIN monomer was immobilized on the streptavidin chip and the MAGE-A3-specific TCR mutant proteins were flowed through the chip. B. Determination of 3D affinity between MAGE-A3-specific TCR mutants and HLA-A1-TITIN by SPR. Equilibrium curves of MAGE-A3-specific TCR mutants binding to HLA-A1-TITIN pMHC at 25° C. Data shown was measured at equilibrium (black dots). Black lines show the fit to a 1:1 binding curve.

FIG. 19A. Biomembrane force probe experiments to measure bond lifetime force curves for 94a-14 TCR or 20a-18 TCR binding to A1-TITIN. Data are shown as mean±SEM of 500+ individual bond lifetimes per force curve.

Table 1. 3D K_{D} and EC₅₀ of each TCR55b-A50 mutant.

Table 2. 3D K_{D} of selected MAGE TCR mutants.

### DETAILED DESCRIPTION

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Provided are engineered TCRs, sequences encoding such a TCR, and a cell (e.g., a population of cells, such as a population of immune effector cells) engineered to express the engineered antigen receptor. In some embodiments, the immune effector cell of the present invention is a T cell or an NK cell. In some embodiments, the T cell is a CD4+ T cell, a CD8+ T cell, or a combination thereof. In one aspect, the cells of the present invention are human cells.

In some embodiments, the subject has a disease associated with expression of a tumor antigen, e.g., a proliferative disease, a precancerous condition, a cancer, and a non-cancer related indication associated with expression of the tumor antigen. In some embodiments, the subject has a MAGE-A3 expressing cancer, including without limitation melanoma, small cell lung cancer, hematologic malignancies, neoplasms of breast, skin, glioma, neuroblastoma, intestine, colorectal, ovary and the kidney. The present invention provides uses of the compositions and/or methods described here for treatment of cancer.

The present invention further provides a method of manufacturing a TCR-expressing cell, comprising introducing nucleic acid encoding an engineered TCR into a cell such that said nucleic acid integrates into the genome of the cell.

T-cell receptor-engineered T cell adoptive therapy. T-cell receptor (TCR)-engineered T cells are an option for adoptive cell therapy used for the treatment of cancer and other conditions. Adoptive cell therapy using, for example, tumor infiltrating lymphocytes (TILs), e.g. autologous TILs expanded ex vivo, has been used as an effective approach to treat certain cancers. However it can be difficult to obtain a therapeutically useful dose of antigen-specific TILs. Using T-cell receptor (TCR) engineering technologies, T cells can be engineered to express an appropriate therapeutic TCR. Tumor antigen-specific TCR gene-engineered T cells are therefore considered as a potentially “off-the-shelf” treatment for cancer patients.

For example, T cells may be isolated from patient blood or tumor tissue. TCR α and β chains engineered by the methods disclosed herein are provided in a suitable vector, e.g. lentivirus, retrovirus, etc. or gene editing system and used to modify the T cells isolated from the patient to encode the desired TCRαβ sequences. These modified T cells are then expanded in vitro to obtain sufficient numbers for treatment and re-infused back into the patient. Alternatively, allogeneic T cells can be used for this purpose. TCR engineered T cells can target and kill cancer cells expressing appropriate antigens.

Cells for use in the methods as described above may be collected from a subject or a donor may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

Techniques for affinity separation may include magnetic separation, using antibody-coated magnetic beads, affinity chromatography, cytotoxic agents joined to a monoclonal antibody or used in conjunction with a monoclonal antibody, e.g., complement and cytotoxic cells, and “panning” with antibody attached to a solid matrix, e.g., a plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g., propidium iodide). Any technique may be employed which is not unduly detrimental to the viability of the selected cells. The affinity reagents may be specific receptors or ligands for the cell surface molecules indicated above. In addition to antibody reagents, peptide-MHC antigen and T cell receptor pairs may be used; peptide ligands and receptor; effector and receptor molecules, and the like.

The separated cells may be collected in any appropriate medium that maintains the viability of the cells, usually having a cushion of serum at the bottom of the collection tube. Various media are commercially available and may be used according to the nature of the cells, including dMEM, HBSS, dPBS, RPMI, Iscove's medium, etc., frequently supplemented with fetal calf serum (FCS).

The collected and optionally enriched cell population may be used immediately for genetic modification, or may be frozen at liquid nitrogen temperatures and stored, being thawed and capable of being reused. The cells will usually be stored in 10% DMSO, 50% FCS, 40% RPMI 1640 medium.

The engineered cells may be infused to the subject in any physiologically acceptable medium by any convenient route of administration, normally intravascularly, although they may also be introduced by other routes, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

Many tumor-specific antigens have been identified. These antigens can induce an immune responses and are promising candidate targets for use in vaccination or T cell therapy, such as melanoma-associated antigen (MAGE)-A3, MAGE-A4, New York esophageal squamous cell carcinoma (NY-ESO)-1, MART-1, gp100, carcino-embryonic antigen (CEA), p53, neoantigens, and the like.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “stimulation,” refers to a primary response induced by binding of a stimulatory molecule (e.g., a TCR/CD3 complex) with its cognate ligand (or tumor antigen in the case of a TCR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM.

The term a “costimulatory molecule” refers to a cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are contribute to an efficient immune response. Costimulatory molecules include, but are not limited to an MHC class I molecule, BTLA and a Toll ligand receptor, as well as OX40, CD27, CD28, CDS, ICAM-1, LFA-1 (CD11a/CD18), ICOS (CD278), and 4-1BB (CD137).

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and macrophages.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell.

The term “effective amount” or “therapeutically effective amount” are used interchangeably herein, and refer to an amount of a compound, formulation, material, or composition, as described herein effective to achieve a particular biological result.

The terms “cancer associated antigen” or “tumor antigen” interchangeably refers to a molecule (typically a protein, carbohydrate or lipid) that is expressed on the surface of a cancer cell, either entirely or as a fragment (e.g., MHC/peptide), and which is useful for the preferential targeting of a pharmacological agent to the cancer cell. In some embodiments, a tumor antigen is a marker expressed by both normal cells and cancer cells, e.g., a lineage marker, e.g., CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (e.g., MHC/peptide), and not synthesized or expressed on the surface of a normal cell.

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

MAGE-A3 is a tumor-specific protein, and has been identified on many tumors including melanoma, small cell lung cancer, hematologic malignancies, neoplasms of breast, skin, glioma, neuroblastoma, intestine, colorectal, ovary and the kidney and others; it is silent in all normal human tissues with the exception of testis and placenta. The human protein refseq can be accessed at NP_005353. See, for example, Saiag et al. Prospective assessment of a gene signature potentially predictive of clinical benefit in metastatic melanoma patients following MAGE-A3 immunotherapeutic (PREDICT). Ann Oncol. 2016; 27:1947-1953; Pujol et al. Safety and Immunogenicity of MAGE-A3 cancer immunotherapeutic with or without adjuvant chemotherapy in patients with resected stage IB to III MAGE-A3-positive non-small-cell lung cancer. J Thorac Oncol. 2015; 10:1458-1467.

MHC context. The function of MHC molecules is to bind peptide fragments derived from pathogens or aberrant proteins derived from transformed cells, and display them on the cell surface for recognition by the appropriate T cells. Thus, T cell receptor recognition can be influenced by the MHC protein that is presenting the antigen. The term MHC context refers to the recognition by a TCR of a given peptide, when it is presented by a specific MHC protein.

Peptide ligands are peptide antigens against which an immune response involving T lymphocyte antigen specific response can be generated. Such antigens include antigens associated with autoimmune disease, infection, cancer neoantigens, foodstuffs such as gluten, etc., allergy or tissue transplant rejection. Antigens also include various microbial antigens, e.g. as found in infection, in vaccination, etc., including but not limited to antigens derived from virus, bacteria, fungi, protozoans, parasites and tumor cells. Tumor antigens include tumor specific antigens, e.g. immunoglobulin idiotypes and T cell antigen receptors; oncogenes, such as p21/ras, p53, p210/bcr-abl fusion product; etc.; developmental antigens, e.g. MART-1/Melan A; MAGE-1, MAGE-3; GAGE family; telomerase; etc.; viral antigens, e.g. human papilloma virus, Epstein Barr virus, etc.; tissue specific self-antigens, e.g. tyrosinase; gp100; prostatic acid phosphatase, prostate specific antigen, prostate specific membrane antigen; thyroglobulin, α-fetoprotein; etc.; and self-antigens, e.g. her-2/neu; carcinoembryonic antigen, muc-1, and the like.

MHC proteins include any of the mammalian MHC proteins. Human HLA proteins are of interest, particularly HLA Class I proteins, e.g. human HLA-A, HLA-B, HLA-C. As will be appreciated by one with skill in the art, the HLA locus is highly polymorphic and a large number of sequence variants are known and described in the art, including without limitation any of the HLA-A*01, HLA-A*02, up to HLA-A*80 alleles and serotypes thereof; and the HLA-B*07, HLA-B*08 up to HLA-B*83 and serotypes thereof. For some embodiments HLA Class II proteins are of interest, e.g. HLA-DPA1, HLA-DPB1, HLA-DQA1, HLA-DQB1, HLA-DRA, and HLA-DRB1. MHC sequences used for screening purposes typically comprise the peptide binding region, e.g. the alpha 1 and alpha 2 domains, or the portion of those domains required to form a peptide binding complex, complexes with a peptide antigen.

Catch bonds are receptor-ligand bonds whose lifetime increases with tensile force applied to the bond (in contrast to the more prevalent slip bonds, whose lifetime is shortened by tensile forces acting on the bond). For example, a ligand-binding domain may be in close contact with a neighboring regulatory domain distal to the binding pocket. Application of a tensile force to the ligand-receptor complex leads to a structural loosening of the interface between the binding pocket and the regulatory domain that activates the binding pocket. Thus, at least two structural states of the receptor-ligand complex can coexist: a short-lived and a long-lived state, each of which has a distinct ligand on- and off-rate. Mechanical perturbations at the domain-domain interface can propagate rapidly to the binding pocket to switch it into the long lived state.

It has often been shown that mechanical force only accelerates the activation of catch bonds, since the short- and long-lived states are separated by an energy barrier that can also be overcome by other means, for example by thermal activation. The role of force is thus only to accelerate the process of passing from the short- to the long-lived state across the energy barrier.

The terms “cancer”, “neoplasm”, “tumor”, and “carcinoma”, are used interchangeably herein to refer to cells which exhibit relatively autonomous growth, so that they exhibit an aberrant growth phenotype characterized by a significant loss of control of cell proliferation. In general, cells of interest for detection or treatment in the present application include precancerous (e.g., benign), malignant, pre-metastatic, metastatic, and non-metastatic cells. Detection of cancerous cells is of particular interest. The term “normal” as used in the context of “normal cell,” is meant to refer to a cell of an untransformed phenotype or exhibiting a morphology of a non-transformed cell of the tissue type being examined. “Cancerous phenotype” generally refers to any of a variety of biological phenomena that are characteristic of a cancerous cell, which phenomena can vary with the type of cancer. The cancerous phenotype is generally identified by abnormalities in, for example, cell growth or proliferation (e.g., uncontrolled growth or proliferation), regulation of the cell cycle, cell mobility, cell-cell interaction, or metastasis, etc.

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

The term “anti-cancer effect” refers to a biological effect which can be manifested by various means, including but not limited to, e.g., a decrease in tumor volume, a decrease in the number of cancer cells, a decrease in the number of metastases, an increase in life expectancy, decrease in cancer cell proliferation, decrease in cancer cell survival, or amelioration of various physiological symptoms associated with the cancerous condition. An “anti-cancer effect” can also be manifested by the ability of the engineered cells in prevention of the occurrence of cancer in the first place. The term “anti-tumor effect” refers to a biological effect which can be manifested by various means, including but not limited to, e.g., a decrease in tumor volume, a decrease in the number of tumor cells, a decrease in tumor cell proliferation, or a decrease in tumor cell survival.

The phrase “disease associated with expression of a tumor antigen as described herein” includes, but is not limited to, a disease associated with expression of a tumor antigen as described herein or condition associated with cells which express a tumor antigen as described herein including, e.g., proliferative diseases such as a cancer or malignancy or a precancerous condition such as a myelodysplasia, a myelodysplastic syndrome or a preleukemia; or a noncancer related indication associated with cells which express a tumor antigen as described herein. In one aspect, a cancer associated with expression of a tumor antigen as described herein is a hematological cancer. In one aspect, a cancer associated with expression of a tumor antigen as described herein is a solid cancer. Further diseases associated with expression of a tumor antigen described herein include, but not limited to, e.g., atypical and/or non-classical cancers, malignancies, precancerous conditions or proliferative diseases associated with expression of a tumor antigen as described herein. Non-cancer related indications associated with expression of a tumor antigen as described herein include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels or reduced levels. In an embodiment, the tumor antigen-expressing cells produced detectable levels of a tumor antigen protein at one point, and subsequently produced substantially no detectable tumor antigen protein.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

Expression construct: The coding sequences may be introduced on an expression vector into a cell to be engineered. For example, a coding sequence may be introduced into a target cell using CRISPR technology. CRISPR/Cas9 system can be directly applied to human cells by transfection with a plasmid that encodes Cas9 and sgRNA. The viral delivery of CRISPR components has been extensively demonstrated using lentiviral and retroviral vectors. Gene editing with CRISPR encoded by non-integrating virus, such as adenovirus and adenovirus-associated virus (AAV), has also been reported. Recent discoveries of smaller Cas proteins have enabled and enhanced the combination of this technology with vectors that have gained increasing success for their safety profile and efficiency, such as AAV vectors. The engineered TCR sequences may replace endogenous TCR sequences, or endogenous sequences may otherwise be inactivated.

The nucleic acid encoding a TCR sequence is inserted into a vector for expression and/or integration. Many such vectors are available. The vector components generally include, but are not limited to, one or more of the following: an origin of replication, one or more marker genes, an enhancer element, a promoter, and a transcription termination sequence. Vectors include viral vectors, plasmid vectors, integrating vectors, and the like, e.g. lentiviral vectors, adenoviral and AAV vectors, retroviral vectors, and the like.

Expression vectors may contain a selection gene, also termed a selectable marker. This gene encodes a protein necessary for the survival or growth of transformed host cells grown in a selective culture medium or a truncated gene encoding a surface marker that allows for antibody based detection. Host cells not transformed with the vector containing the selection gene will not survive in the culture medium. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, or (d) enable surface antibody based detection for isolation via fluoresences activating cell sorting (FACS) or magnetic separation e.g. truncated forms of NGFR, EGFR, CD19.

Nucleic acids are “operably linked” when placed into a functional relationship with another nucleic acid sequence. For example, DNA for a signal sequence is operably linked to DNA for a polypeptide if it is expressed as a preprotein that signals the secretion of the polypeptide; a promoter or enhancer is operably linked to a coding sequence if it affects the transcription of the sequence; and a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

Expression vectors will contain a promoter that is recognized by the host organism and is operably linked to the construct coding sequence. Promoters are untranslated sequences located upstream (5′) to the start codon of a structural gene (generally within about 100 to 1000 bp) that control the transcription and translation of particular nucleic acid sequence to which they are operably linked. Such promoters typically fall into two classes, inducible and constitutive. Inducible promoters are promoters that initiate increased levels of transcription from DNA under their control in response to some change in culture conditions, e.g., the presence or absence of a nutrient or a change in temperature. A large number of promoters recognized by a variety of potential host cells are well known.

Transcription from vectors in mammalian host cells may be controlled, for example, by promoters obtained from the genomes of viruses such as polyoma virus, fowlpox virus, adenovirus (such as Adenovirus 2), bovine papilloma virus, avian sarcoma virus, cytomegalovirus, a retrovirus LTR (such as murine stem cell virus), hepatitis-B virus and Simian Virus 40 (SV40), from heterologous mammalian promoters, e.g., the actin promoter, PGK (phosphoglycerate kinase), or an immunoglobulin promoter, or from heat-shock promoters, provided such promoters are compatible with the host cell systems. The early and late promoters of the SV40 virus are conveniently obtained as an SV40 restriction fragment that also contains the SV40 viral origin of replication.

Transcription by higher eukaryotes may be increased by inserting an enhancer sequence into the vector. Enhancers are cis-acting elements of DNA, usually about from 10 to 300 bp in length, which act on a promoter to increase its transcription. Enhancers are relatively orientation and position independent, having been found 5′ and 3′ to the transcription unit, within an intron, as well as within the coding sequence itself. Many enhancer sequences are now known from mammalian genes (globin, elastase, albumin, α-fetoprotein, and insulin). Typically, however, one will use an enhancer from a eukaryotic virus. Examples include the SV40 enhancer on the late side of the replication origin, the cytomegalovirus early promoter enhancer, the polyoma enhancer on the late side of the replication origin, and adenovirus enhancers. The enhancer may be spliced into the expression vector at a position 5′ or 3′ to the coding sequence, but is preferably located at a site 5′ from the promoter.

Expression vectors for use in eukaryotic host cells will also contain sequences necessary for the termination of transcription and for stabilizing the mRNA. Such sequences are commonly available from the 5′ and, occasionally 3′, untranslated regions of eukaryotic or viral DNAs or cDNAs. Construction of suitable vectors containing one or more of the above-listed components employs standard techniques.

Suitable host cells for cloning a construct are the prokaryotic, yeast, or other eukaryotic cells described above. Examples of useful mammalian host cell lines are mouse L cells (L-M[K−], ATCC #CRL-2648), monkey kidney CV1 line transformed by SV40 (COS-7, ATCC CRL 1651); human embryonic kidney line (293 or 293 cells subcloned for growth in suspension culture; baby hamster kidney cells (BHK, ATCC CCL 10); Chinese hamster ovary cells/-DHFR (CHO); mouse Sertoli cells (TM4); monkey kidney cells (CV1 ATCC CCL 70); African green monkey kidney cells (VERO-76, ATCC CRL-1 587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells; MRC 5 cells; FS4 cells; and a human hepatoma line (Hep G2).

Host cells, including T cells, stem cells, etc. can be transfected with the above-described expression vectors for construct expression. Cells may be cultured in conventional nutrient media modified as appropriate for inducing promoters, selecting transformants, or amplifying the genes encoding the desired sequences. Mammalian host cells may be cultured in a variety of media. Commercially available media such as Ham's F10 (Sigma), Minimal Essential Medium ((MEM), Sigma), RPMI 1640 (Sigma), and Dulbecco's Modified Eagle's Medium ((DMEM), Sigma) are suitable for culturing the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics, trace elements, and glucose or an equivalent energy source. Any other necessary supplements may also be included at appropriate concentrations that would be known to those skilled in the art. The culture conditions, such as temperature, pH and the like, are those previously used with the host cell selected for expression, and will be apparent to the ordinarily skilled artisan.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a TCR of the invention can be replaced with other amino acid residues from the same side chain family and the altered TCR can be tested using the functional assays described herein.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “sequence identity,” as used herein in reference to polypeptide or DNA sequences, refers to the subunit sequence identity between two molecules. When a subunit position in both of the molecules is occupied by the same monomeric subunit (e.g., the same amino acid residue or nucleotide), then the molecules are identical at that position. The similarity between two amino acid or two nucleotide sequences is a direct function of the number of identical positions. In general, the sequences are aligned so that the highest order match is obtained. If necessary, identity can be calculated using published techniques and widely available computer programs, such as the GCS program package (Devereux et al., Nucleic Acids Res. 12:387, 1984), BLASTP, BLASTN, FASTA (Atschul et al., J. Molecular Biol. 215:403, 1990).

“Derived from” as that term is used herein, indicates a relationship between a first and a second molecule. It generally refers to structural similarity between the first molecule and a second molecule and does not connotate or include a process or source limitation on a first

By “protein variant” or “variant protein” or “variant polypeptide” herein is meant a protein that differs from a wild-type protein by virtue of at least one amino acid modification. The parent polypeptide may be a naturally occurring or wild-type (WT) polypeptide, or may be a modified version of a WT polypeptide. Variant polypeptide may refer to the polypeptide itself, a composition comprising the polypeptide, or the amino sequence that encodes it. Preferably, the variant polypeptide has at least one amino acid modification compared to the parent polypeptide, e.g. from about one to about ten amino acid modifications, and preferably from about one to about five amino acid modifications compared to the parent.

By “parent polypeptide”, “parent protein”, “precursor polypeptide”, or “precursor protein” as used herein is meant an unmodified polypeptide that is subsequently modified to generate a variant. A parent polypeptide may be a wild-type (or native) polypeptide, or a variant or engineered version of a wild-type polypeptide. Parent polypeptide may refer to the polypeptide itself, compositions that comprise the parent polypeptide, or the amino acid sequence that encodes it.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, gamma-carboxyglutamate, and O-phosphoserine. “Amino acid analogs” refers to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid.

Amino acid modifications disclosed herein may include amino acid substitutions, deletions and insertions, particularly amino acid substitutions. Variant proteins may also include conservative modifications and substitutions at other positions of the cytokine and/or receptor (e.g., positions other than those involved in the affinity engineering). Such conservative substitutions include those described by Dayhoff in The Atlas of Protein Sequence and Structure 5 (1978), and by Argos in EMBO J., 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, lie, Leu, Met, Ala, Phe; Group IV: Lys, Arg, His; Group V: Phe, Tyr, Trp, His; and Group VI: Asp, Glu. Further, amino acid substitutions with a designated amino acid may be replaced with a conservative change.

The term “isolated” refers to a molecule that is substantially free of its natural environment. For instance, an isolated protein is substantially free of cellular material or other proteins from the cell or tissue source from which it is derived. The term refers to preparations where the isolated protein is sufficiently pure to be administered as a therapeutic composition, or at least 70% to 80% (w/w) pure, more preferably, at least 80%-90% (w/w) pure, even more preferably, 90-95% pure; and, most preferably, at least 95%, 96%, 97%, 98%, 99%, or 100% (w/w) pure. A “separated” compound refers to a compound that is removed from at least 90% of at least one component of a sample from which the compound was obtained. Any compound described herein can be provided as an isolated or separated compound.

Library. In some embodiments of the invention, a library is provided of polypeptides, or of nucleic acids encoding such polypeptides, usually a library of different TCR modified at one or more residues of the CDR loops. Conventional methods of assembling the coding sequences can be used. In order to generate the diversity of sequences, randomization, error prone PCR, mutagenic primers, and the like as known in the art, are used to create a set of polynucleotides. The library of polynucleotides is typically ligated to a vector suitable for the host cell of interest. In various embodiments the library is provided as a purified polynucleotide composition encoding polypeptides, where the population of cells can be, without limitation mammalian T cells, and where the cells are induced to express the polypeptide library.

“Suitable conditions” shall have a meaning dependent on the context in which this term is used. That is, when used in connection with binding of a T cell receptor to a pMHC complex, the term shall mean conditions that permit a TCR to bind to a cognate peptide ligand. When this term is used in connection with nucleic acid hybridization, the term shall mean conditions that permit a nucleic acid of at least 15 nucleotides in length to hybridize to a nucleic acid having a sequence complementary thereto. When used in connection with contacting an agent to a cell, this term shall mean conditions that permit an agent capable of doing so to enter a cell and perform its intended function. In one embodiment, the term “suitable conditions” as used herein means physiological conditions.

The terms “subject,” “individual,” and “patient” are used interchangeably herein to refer to a mammal being assessed for treatment and/or being treated. In some embodiments, the mammal is a human. The terms “subject,” “individual,” and “patient” encompass, without limitation, individuals having a disease. Subjects may be human, but also include other mammals, particularly those mammals useful as laboratory models for human disease, e.g., mice, rats, etc.

The term “sample” with reference to a patient encompasses blood and other liquid samples of biological origin, solid tissue samples such as a biopsy specimen or tissue cultures or cells derived therefrom and the progeny thereof. The term also encompasses samples that have been manipulated in any way after their procurement, such as by treatment with reagents; washed; or enrichment for certain cell populations, such as diseased cells. The definition also includes samples that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's diseased cell, e.g., a sample comprising polynucleotides and/or polypeptides that is obtained from a patient's diseased cell (e.g., a cell lysate or other cell extract comprising polynucleotides and/or polypeptides); and a sample comprising diseased cells from a patient. A biological sample comprising a diseased cell from a patient can also include non-diseased cells.

The term “diagnosis” is used herein to refer to the identification of a molecular or pathological state, disease or condition in a subject, individual, or patient.

The term “prognosis” is used herein to refer to the prediction of the likelihood of death or disease progression, including recurrence, spread, and drug resistance, in a subject, individual, or patient. The term “prediction” is used herein to refer to the act of foretelling or estimating, based on observation, experience, or scientific reasoning, the likelihood of a subject, individual, or patient experiencing a particular event or clinical outcome. In one example, a physician may attempt to predict the likelihood that a patient will survive.

As used herein, the terms “treatment,” “treating,” and the like, refer to administering an agent, or carrying out a procedure, for the purposes of obtaining an effect on or in a subject, individual, or patient. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of cancer in a mammal, particularly in a human, and includes: (a) inhibiting the disease, i.e., arresting its development; and (b) relieving the disease or its symptoms, i.e., causing regression of the disease or its symptoms.

Treating may refer to any indicia of success in the treatment or amelioration or prevention of a disease, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of engineered cells to prevent or delay, to alleviate, or to arrest or inhibit development of the symptoms or conditions associated with disease or other diseases. The term “therapeutic effect” refers to the reduction, elimination, or prevention of the disease, symptoms of the disease, or side effects of the disease in the subject.

As used herein, a “therapeutically effective amount” refers to that amount of the therapeutic agent sufficient to treat or manage a disease or disorder. A therapeutically effective amount may refer to the amount of therapeutic agent sufficient to delay or minimize the onset of disease, e.g., to delay or minimize the growth and spread of cancer. A therapeutically effective amount may also refer to the amount of the therapeutic agent that provides a therapeutic benefit in the treatment or management of a disease. Further, a therapeutically effective amount with respect to a therapeutic agent of the invention means the amount of therapeutic agent alone, or in combination with other therapies, that provides a therapeutic benefit in the treatment or management of a disease.

As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MED14736, MPDL3280A and MSB0010718C targeting PD-L1 and those targeting CTLA-4 such as ipilimumab.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

### Engineered T Cell Receptor and Cell Compositions

Polypeptide constructs and compositions are provided, which comprise a engineered TCR sequence. In some embodiments the engineered TCR is specific for MAGE-A3, and comprises an alpha chain (TCRα) of SEQ ID NO:1, or a mature protein thereof, i.e. lacking the signal sequence of residues 1-18, comprising at least one amino acid modification to enhance target activation potency at one or more residues selected from D28, A30, I51, Q52, S53 and S54 (numbering relative to the mature protein sequence). In some embodiments the amino acid modification is an amino acid substitution. In some embodiments the amino acid substitution is selected from D28H/N/G/K/S; A30H/S/E/N/G; I51V; Q52R/H; S53P; S54Y/N/R/E/D/H. In some embodiments the TCRα has a sequence selected from SEQ ID NO:2-SEQ ID NO:15, or a variant derived therefrom. Variants may comprise at least about 90% sequence identity, at least 95% sequence identity, at least about 97%, sequence identity, at least about 99% sequence identity to a reference sequence of SEQ ID NO:2-15. In any of such embodiments the beta chain (TCR3) may have the sequence set forth in SEQ ID NO:16. The MAGE-A3 engineered TCR does not have significant affinity for human titin sequences.

In some embodiments the engineered TCR is specific for HIV peptide presented by HLA-B35, based on amino acid modifications of TCR55 alpha chain (SEQ ID NO:17) and TCR55 beta chain (SEQ ID NO:18). The amino acid modifications include, without limitation, SEQ ID NO:17 A98D, A98E, A98F, A98Q, A98Y, A98H to make TCR55 activated by B35-HIV. Amino acid modification in TCR55 beta chain (SEQ ID NO:18) include, without limitation, A50D, A50E, A50F, A50H, A50N, A500, A50S, A50T, A50Y to make TCR55 activated by B35-HIV.

An engineered TCR, e.g. a TCR specific for MAGE-A3, may have a 3D log K_{D} (μM) of from about 0.5 to about 100 μM, and may be from about 1 to about 100 μM, from about 1 to about 50 μM. “Affinity” refers to the strength of binding, increased binding affinity being correlated with a lower K_{D}. In an embodiment, affinity is determined by surface plasmon resonance (SPR), e.g. as used by Biacore systems. The affinity of one molecule for another molecule is determined by measuring the binding kinetics of the interaction, e.g. at 25° C. The engineered TCR is desirably selected for target activation potency, as measured by any convenient assay, including without limitation T cell proliferation in response to antigen, release of IL-2 in response to antigen, upregulation of CD69 on a T cell in response to antigen, and the like.

Also provided are isolated nucleic acids encoding the engineered TCR sequence and constructs thereof, vectors and host cells comprising the nucleic acid, and recombinant techniques for the production of the polypeptide constructs. Nucleic acids of interest encode a polypeptide that is at least about 80% identical to the provided polypeptide sequences, at least about 85%, at least about 90%, at least about 95%, at least about 99%, or identical. Polynucleotide sequences may encode any or all of the provided sequences.

In some embodiments, a vector comprising a coding sequence that encodes engineered TCR sequence or engineered TCR construct is provided, where the coding sequence is operably linked to a promoter active in the desired cell; or is provided in a vector suitable for genomic insertion, e.g., by CRISPR. Various vectors are known in the art and can be used for this purpose, e.g., viral vectors, plasmid vectors, minicircle vectors, which vectors can be integrated into the target cell genome, or can be episomally maintained.

In another embodiment of the invention, an article of manufacture containing an isolated polypeptide or polynucleotide is provided. The article of manufacture comprises a container and a label. Suitable containers include, for example, bottles, vials, syringes, and test tubes. The containers may be formed from a variety of materials such as glass or plastic. The container holds a polypeptide or polynucleotide composition, which may be a therapeutic composition, e.g. for treatment of cancer, and may have a sterile access port (for example the container may be an intravenous solution bag or a vial having a stopper pierceable by a hypodermic injection needle). A label on or associated with the container may indicate that the composition is used for treating the condition of choice. Further container(s) may be provided with the article of manufacture which may hold, for example, a pharmaceutically-acceptable buffer, such as phosphate-buffered saline, Ringer's solution or dextrose solution. The article of manufacture may further include other materials desirable from a commercial and user standpoint, including other buffers, diluents, filters, needles, syringes, and package inserts with instructions for use.

In some embodiments a cell composition is provided. The cell can be provided in a unit dose for therapy, and can be allogeneic, autologous, etc. with respect to an intended recipient. Methods may include a step of obtaining desired cells, e.g., T cells, hematopoietic stem cells, etc., which may be isolated from a biological sample, or may be derived in vitro from a source of progenitor cells. The cells are transduced or transfected with a vector comprising a sequence encoding the engineered TCR, which step may be performed in any suitable culture medium. For example, cells may be collected from a patient, modified ex vivo, and reintroduced into the subject. The cells collected from the subject may be collected from any convenient and appropriate source, including e.g., peripheral blood (e.g., the subject's peripheral blood), a biopsy (e.g., a biopsy from the subject), and the like.

Where the use of autologous cells is not desirable, e.g. where a patient has insufficient T cells for modification, where there is insufficient time to expand autologous cells, etc., allogeneic cells may be used, e.g. T cells or stem cells from a healthy donor. Such allogeneic cells can be genetically modified to reduce GVHD, to reduce host versus graft responses, etc.

Engineered cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

Effector T cells include autologous or allogeneic immune cells having cytolytic activity against a target cell expressing an antigen of interest. The effector cells have cytolytic activity through recognition by the T cell antigen receptor. The term “T cells” refers to mammalian immune effector cells that may be characterized by expression of CD3 and/or T cell antigen receptor.

In some embodiments, the engineered cells comprise a complex mixture of immune cells, e.g., tumor infiltrating lymphocytes (TILs) isolated from an individual in need of treatment. See, for example, Yang and Rosenberg (2016) Adv Immunol. 130:279-94, “Adoptive T Cell Therapy for Cancer; Feldman et al (2015) Semin Oncol. 42(4):626-39 “Adoptive Cell Therapy-Tumor-Infiltrating Lymphocytes, T-Cell Receptors, and Chimeric Antigen Receptors”; Clinical Trial NCT01174121, “Immunotherapy Using Tumor Infiltrating Lymphocytes for Patients With Metastatic Cancer”; Tran et al. (2014) Science 344(6184)641-645, “Cancer immunotherapy based on mutation-specific CD4+ T cells in a patient with epithelial cancer”.

In other embodiments, the engineered T cell is allogeneic with respect to the individual that is treated. See for review Graham et al. (2018) Cells. 7(10) E155. In some embodiments an allogeneic engineered T cell is fully HLA matched. However not all patients have a fully matched donor and a cellular product suitable for all patients independent of HLA type provides an alternative. A universal ‘off the shelf’ T cell product provides advantages in uniformity of harvest and manufacture.

T cells can be genetically modified. For example, the endogenous TCRαβ receptor can be knocked out by different gene editing techniques. TCRαβ is a heterodimer and both alpha and beta chains need to be present for it to be expressed. A single gene codes for the alpha chain (TRAC), whereas there are 2 genes coding for the beta chain, therefore TRAC loci KO has been deleted for this purpose. A number of different approaches have been used to accomplish this deletion, e.g. CRISPR/Cas9; meganuclease; engineered I-Crel homing endonuclease, etc.

Allogeneic T cells may be administered in combination with intensification of lymphodepletion to allow the engineered T cells to expand and clear malignant cells prior to host immune recovery, e.g. by administration of Alemtuzumab (monoclonal anti-CD52), purine analogs, etc. The allogeneic T cells may be modified for resistance to Alemtuzumab, and currently in clinical trials. Gene editing has also been used to prevent expression of HLA class I molecules on CAR-T cells, e.g. by deletion of 32-microglobulin, see NCT03166878.

T cells for engineering as described above collected from a subject or a donor may be separated from a mixture of cells by techniques that enrich for desired cells, or may be engineered and cultured without separation. An appropriate solution may be used for dispersion or suspension. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc.

The cells can be administered by any suitable means, usually parenteral. Parenteral infusions include intramuscular, intravenous (bolus or slow drip), intraarterial, intraperitoneal, intrathecal or subcutaneous administration.

Engineered cells can be provided in pharmaceutical compositions suitable for therapeutic use, e.g. for human treatment. Therapeutic formulations comprising such cells can be frozen, or prepared for administration with physiologically acceptable carriers, excipients or stabilizers (Remington's Pharmaceutical Sciences 16th edition, Osol, A. Ed. (1980)), in the form of aqueous solutions. The cells will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

### Methods of Treatment

The engineered T cells may be infused to the subject in any physiologically acceptable medium, normally intravascularly, although they may also be introduced into any other convenient site, where the cells may find an appropriate site for growth. Usually, at least 1×10⁶ cells/kg will be administered, at least 1×10⁷ cells/kg, at least 1×10⁸ cells/kg, at least 1×10⁹ cells/kg, at least 1×10¹⁰ cells/kg, or more, usually being limited by the number of T cells that are obtained during collection.

For example, typical ranges for the administration of cells for use in the practice of the present invention range from about 1×10¹ to 5×10⁸ viable cells per kg of subject body weight per course of therapy. Consequently, adjusted for body weight, typical ranges for the administration of viable cells in human subjects ranges from approximately 1×10⁶ to approximately 1×10¹³ viable cells, alternatively from approximately 5×10⁶ to approximately 5×10¹² viable cells, alternatively from approximately 1×10¹ to approximately 1×10¹² viable cells, alternatively from approximately 5×10⁷ to approximately 1×10¹² viable cells, alternatively from approximately 1×10⁸ to approximately 1×10¹² viable cells, alternatively from approximately 5×10⁸ to approximately 1×10¹² viable cells, alternatively from approximately 1×10⁹ to approximately 1×10¹² viable cells per course of therapy. In one embodiment, the dose of the cells is in the range of 2.5-5×10⁹ viable cells per course of therapy.

A course of therapy may be a single dose or in multiple doses over a period of time. In some embodiments, the cells are administered in a single dose. In some embodiments, the cells are administered in two or more split doses administered over a period of 1, 2, 3, 4, 5, 6, 7, 8, 9, 10, 11, 12, 13, 14, 21, 28, 30, 60, 90, 120 or 180 days. The quantity of engineered cells administered in such split dosing protocols may be the same in each administration or may be provided at different levels. Multi-day dosing protocols over time periods may be provided by the skilled artisan (e.g. physician) monitoring the administration of the cells taking into account the response of the subject to the treatment including adverse effects of the treatment and their modulation as discussed above.

In one embodiment, the present invention provides a method of treating a subject suffering from a disease, disorder or condition amendable to treatment with adoptive T cell therapy (e.g. cancer) by the administration of an effective dose of the engineered cells disclosed herein. In one embodiment, the present invention provides for a method of treatment of a mammalian subject suffering from a disease, disorder associated with the presence of an aberrant population of cells (e.g. a tumor) said population of cells characterized by the expression of one or more surface antigens (e.g. tumor antigen(s)), the method comprising the steps of (a) obtaining a biological sample comprising T-cells from the individual; (b) enriching the biological sample for the presence of T-cells; (c) transfecting the T-cells with one or more expression vectors comprising a nucleic acid sequence encoding an engineered TCR (d) expanding the population of the TCR expressing T cells ex vivo; (e) administering a pharmaceutically effective amount of the TCR expressing T cells to the mammal. In one embodiment, the foregoing method is associated with lymphodepletion or immunosuppression of the mammal prior to the initiation of the course of T cell therapy. In another embodiment, the foregoing method is practiced in the absence of lymphodepletion and/or immunosuppression of the mammal.

The preferred formulation for therapeutic use depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™ agarose, cellulose, and the like), polymeric amino acids, amino acid copolymers, and lipid aggregates (such as oil droplets or liposomes).

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG).

Formulations to be used for in vivo administration are typically sterile. Sterilization of the compositions of the present invention may readily accomplished by filtration through sterile filtration membranes.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

In some embodiments the subject compositions, methods and kits are used to enhance a T cell mediated immune response. In some embodiments the immune response is directed towards a condition where it is desirable to deplete or regulate target cells, e.g., cancer cells, infected cells, immune cells involved in autoimmune disease, etc. In some embodiments the condition is cancer. The term “cancer”, as used herein, refers to a variety of conditions caused by the abnormal, uncontrolled growth of cells. Cells capable of causing cancer, referred to as “cancer cells”, possess characteristic properties such as uncontrolled proliferation, immortality, metastatic potential, rapid growth and proliferation rate, and/or certain typical morphological features. A cancer can be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer, and detecting a genotype indicative of a cancer. However, a negative result in one or more of the above detection methods does not necessarily indicate the absence of cancer, e.g., a patient who has exhibited a complete response to a cancer treatment may still have a cancer, as evidenced by a subsequent relapse.

### Methods of Screening

Compositions and methods are provided for mutagenizing and selecting TCR sequences for high signaling activation and low off-target cross-reactivity. In such screening methods, a library is generated comprising amino acid variations at pre-determined amino acid residues on the TCR sequence for optimization. The residues selected for mutagenesis are usually within one or more of the CDR regions of the TCR. A TCRα sequence may be mutagenized and paired with a non-mutagenized TCRβ, or TCRα sequence may be mutagenized and paired with a non-mutagenized TCRβ. The library is introduced into mammalian cells for expression, including mammalian T cell lines. The cells are first selected for low affinity binding to the cognate pMHC, e.g. by binding to labeled pMHC tetramers, multimers, etc., and sorting by flow cytometry, etc., for low affinity binding, e.g. binding at a 3D log K_{D} (μM) of from about 0.1 to about 100 μM.

For example, the MHC may be multimerized to a reagent having a detectable label, e.g. for flow cytometry, mass cytometry, etc. For example, FACS sorting can be used to increase the concentration of the cells of having a peptide ligand binding to the TCR. Techniques include fluorescence activated cell sorters, which can have varying degrees of sophistication, such as multiple color channels, low angle and obtuse light scattering detecting channels, impedance channels, etc.

The low affinity TCR sequences are screened for the ability to activate T cells in response to antigen. Where the initial screening was done in T cells, the T cells may be directly screened; alternatively the sequences of low affinity binding TCR are introduced into T cells for activation screening. The population of T cells expressing TCRs with low antigen affinity are incubated with an antigen source, e.g. target cells expressing the cognate antigen, a pMHC substrate, antigen-presenting cells pulsed with antigenic peptide, etc., for a period of time sufficient to activate the T cells. The T cells are selected for high levels of activation, e.g. by proliferation, IL-2 release, CD69 upregulation, etc. Conveniently, upregulation of CD69 is selected by antibody staining and flow cytometry. Such selection may be based on relative values, where the cells in the top 20%, top 10%, top 5%, top 1% are selected. Rounds of selection are performed until the selected population has a desired level of affinity and activation. Usually at least three and more usually at least four rounds of selection are performed. The resulting engineered TCR may be validated for low off-target cross-reactivity and high on-target activation.

After a final round of selection, polynucleotides are isolated from the selected host cells, and the sequence of the selected TCR are determined, usually by high throughput sequencing. The desired affinity may be at a K_{D} from about 10⁻⁶ M to about 10⁻⁹M.

The peptide sequence results and database search results may be provided in a variety of media to facilitate their use. “Media” refers to a manufacture that contains the expression repertoire information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression repertoire.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

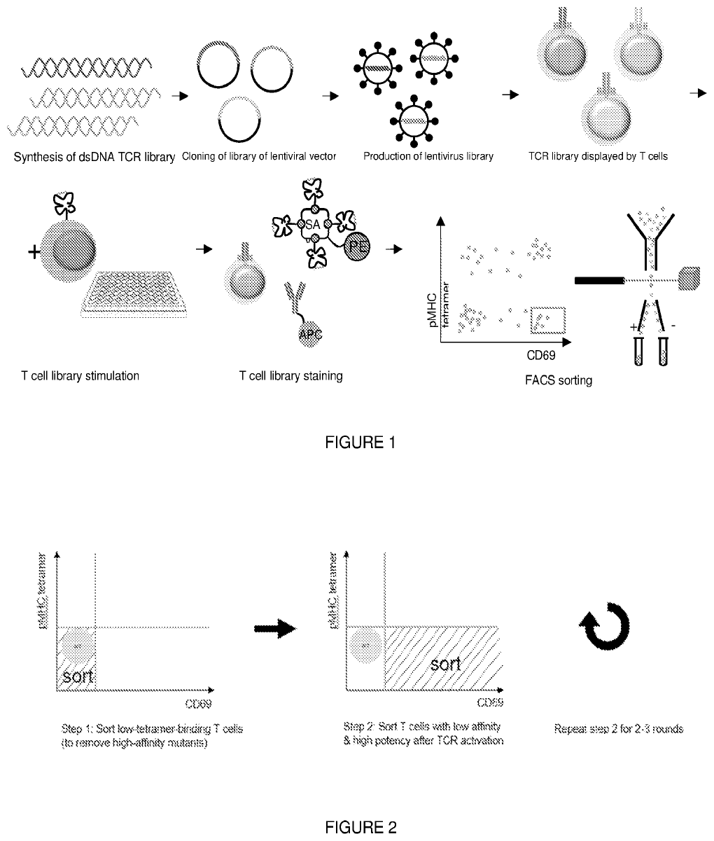

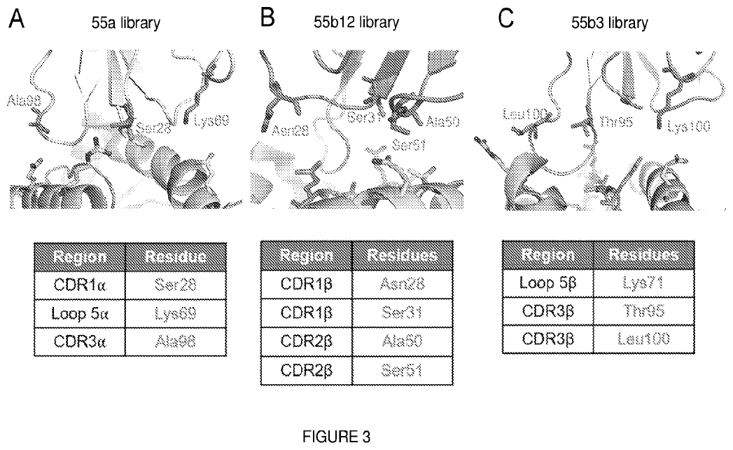

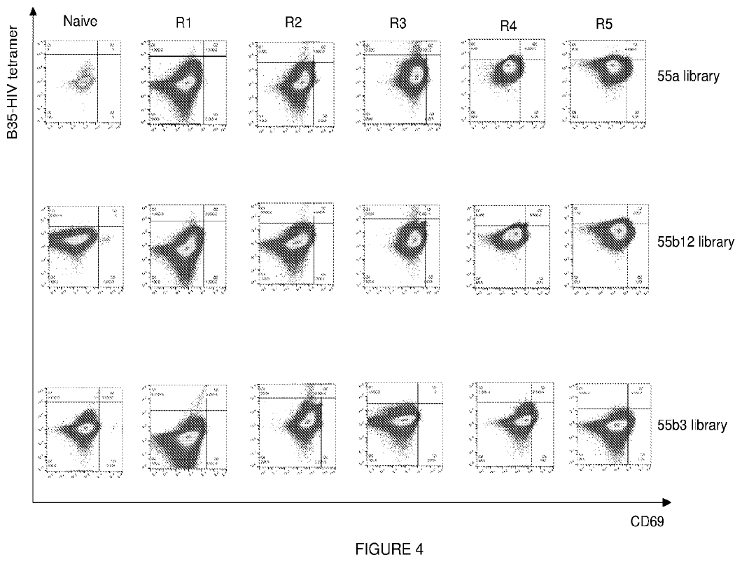

TCR libraries were synthesized as dsDNA with randomized residues. The library was cloned into lentiviral vector by Gibson assembly. The library of recombinant lentiviral vectors were used to produce the library of lentivirus to infect SKW-3 T cell line. The display of TCR library on SKW-3 T cells were detected by anti-TCR (clone IP26) staining. The T cell library was cocultured with antigen-presenting cells pulsed with 10 μM antigenic peptide for 14 hours, and the T cell library was stained with anti-CD69-APC and specific pMHC tetramer. Any clones with high-level anti-CD69 staining and low-level tetramer staining were sorted for further rounds of sorting or analysis, schematic shown in FIG. 1.

In the round 0, the T cell library or WT TCR transfectant was stained with anti-CD69-APC and specific pMHC tetramer. The T cell library clones which have similar level of anti-CD69 and tetramer staining compared to WT TCR transfectants were sorted to remove any high-affinity or auto-responsive clones. Afterwards, the T cell library was cocultured with antigen-presenting cells pulsed with 10 μM antigenic peptide for 14 hours, and the T cell library was stained with anti-CD69-APC and specific pMHC tetramer. Any clones with high-level anti-CD69 staining and low-level tetramer staining were sorted. The same sorting procedure as round 2 were repeated for 2-3 more rounds to further enrich certain mutants.

Design of TCR55 libraries. Based on the structure of B35-HIV-TCR55 (PDB ID: 6BJ3), residues on TCR55 alpha chain (Ser28, Lys69, Ala98) were selected and randomized into VRW codon as 55a library (A); residues on TCR55b beta chain CDR1 and CDR2 (Asn28, Ser31, Ala50 and Ser51) were selected and randomized into VRW codon as 55b12 library (B); residues on TCR55b beta chain CDR3 (Lys71, Thr95 and Leu100) were selected and randomized into VRW codon as 55b3 library) (C).

The TCR55 library was sorted for 5 rounds of selection. In each round, the T cell library was cocultured with KG-1 cells pulsed with 10 μM HIV peptide for 14 hours, and the T cell library was stained with anti-CD69-APC and HLA-B35-HIV tetramer. Any clones with high-level anti-CD69 staining and low-level tetramer staining were sorted. Gating is based on the anti-CD69 and B35-HIV tetramer staining of TCR55 WT transfectants.

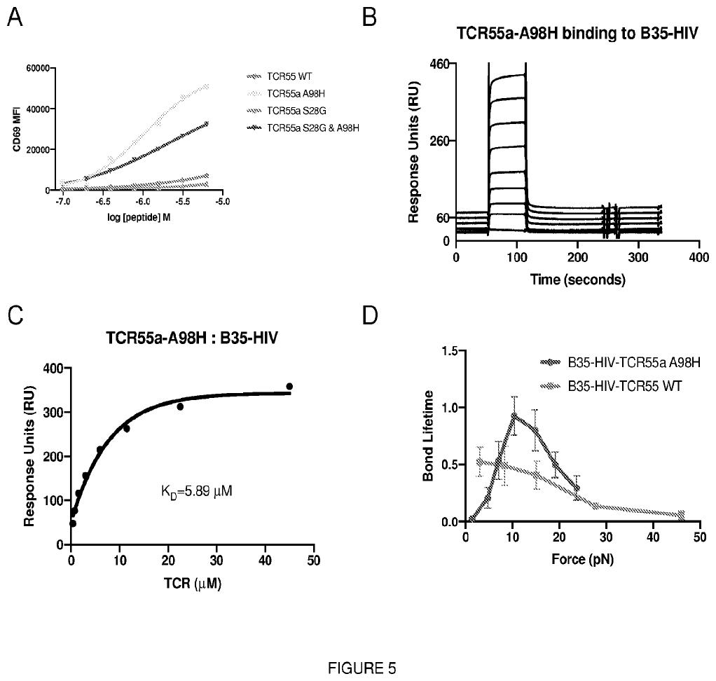

TCR55a-A98H is a catch bond-engineered TCR which can be activated by B35-HIV. Shown in FIG. 5, TCR55 WT, TCR55a-A98H, TCR55a-S28G or TCR55a-S28G A98H T cell transfectants were cocultured with KG-1 cells pulsed with titrated HIV peptide for 14 hours and stained with anti-CD69. The experiment was analyzed by flow cytometry. Surface plasmon resonance (SPR) experiment to measure the 3D binding affinity between immobilized B35-HIV and flowed TCR55a-A98H protein. Biomembrane force probe (BFP) experiment to measure the bond lifetime between B35-HIV protein and TCR55 WT or TCR55a-A98H T cell transfectants.

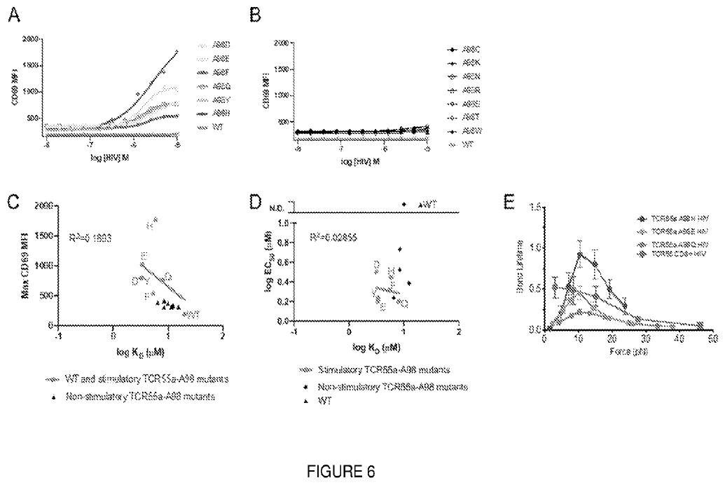

As shown in FIG. 6, TCR55a-Ala98 is a hot spot for catch bond engineering. TCR55a-A98 mutation to D, E, F, Q, Y and H were made as T cell transfectants and stimulated by KG-1 cells pulsed with titrated HIV peptide for 14 hours and stained with anti-CD69. The experiment was analyzed by flow cytometry. B. TCR55a-A98 mutation to C, K, N, R, S, T and W were made as T cell transfectants and stimulated by KG-1 cells pulsed with titrated HIV peptide. Analysis was the same as A. C. The correlation between Emax and 3D binding affinity (K_{D}) of stimulatory TCR55a-A98 mutants. D. The correlation between EC₅₀ and 3D K_{D} of stimulatory TCR55a-A98 mutants. E. BFP measurement of bond lifetime of TCR55a-A98H, TCR55a-A98E and TCR55a-A98Q T cell transfectants interacting with B35-HIV protein.

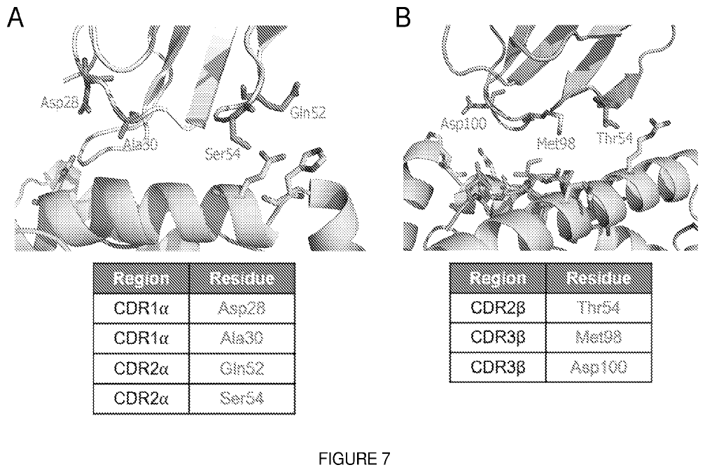

FIG. 7 shows the protein sequence of TCR55 alpha chain (SEQ ID NO:17) and TCR55 beta chain (SEQ ID NO:18). A. The highlight and underlined A in TCR55 alpha chain is the Ala98 hotspot which can be mutated to D, E, F, Q, Y and H to make TCR55 activated by B35-HIV. B. The highlight and underlined A in TCR55 beta chain is the Ala50 hotspot which can be mutated to D, E, F, H, N, Q, S, T and Y to make TCR55 activated by B35-HIV.

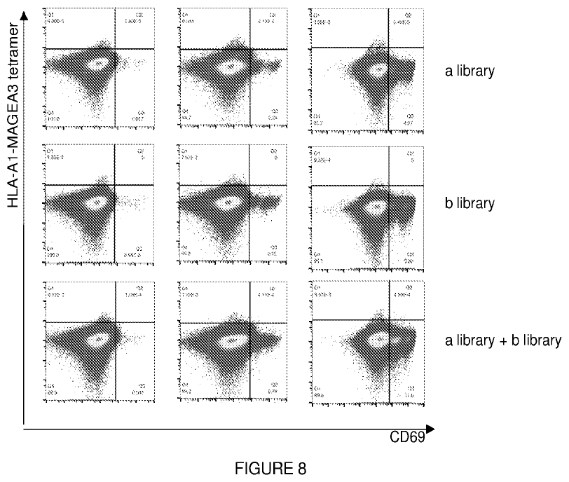

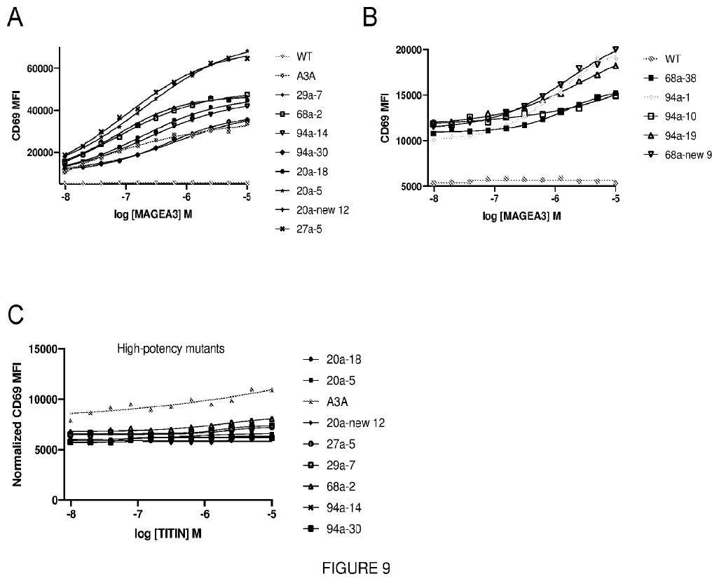

Based on the structure of HLA-A1-MAGEA3-MAG-IC3 (PDB ID: 5BRZ), residues on TCR alpha chain (Asp28, Ala30, Ser54 and Gln52) were selected and randomized into VRW codon as a library (A); residues on TCR beta chain (Thr54, Met98 and Asp100) were selected and randomized into VRW codon as b library (B), shown in FIG. 8.

The MAGE libraries were selected. In each round, the T cell library was cocultured with antigen-presenting cells pulsed with 10 μM MAGEA3 peptide for 14 hours, and the T cell library was stained with anti-CD69-APC and HLA-A1-MAGEA3 tetramer. Any clones with high-level anti-CD69 staining and low-level tetramer staining were sorted. Gating is based on the anti-CD69 and HLA-A1-MAGEA3 tetramer staining of MAGEA3 WT TCR transfectants.

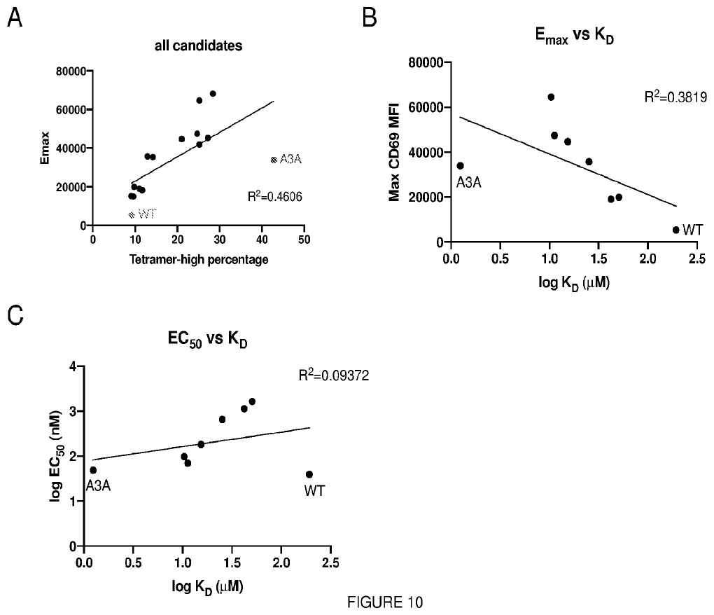

Multiple TCR mutants were identified to be activated by MAGEA3 tumor antigen. 8 high-potency mutants T cells were cocultured with 293T-HLA-A1 cells pulsed with titrated MAGEA3 peptide for 14 hours. T cells were stained with anti-CD69 and analyzed on flow cytometry, shown in FIG. 10. 5 intermediate-potency mutants T cells were cocultured with 293T-HLA-A1 cells pulsed with titrated MAGEA3 peptide for 14 hours. T cells were stained with anti-CD69 and analyzed on flow cytometry. 8 high-potency mutants T cells were cocultured with 293T-HLA-A1 cells pulsed with titrated TITIN peptide for 14 hours. T cells were stained with anti-CD69 and analyzed on flow cytometry.

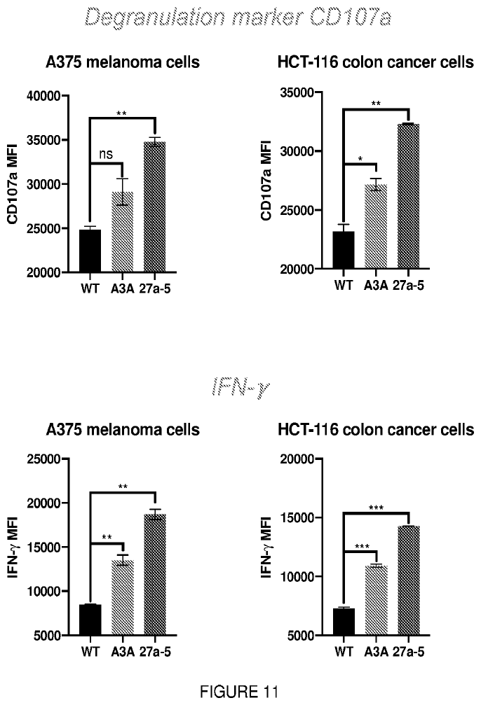

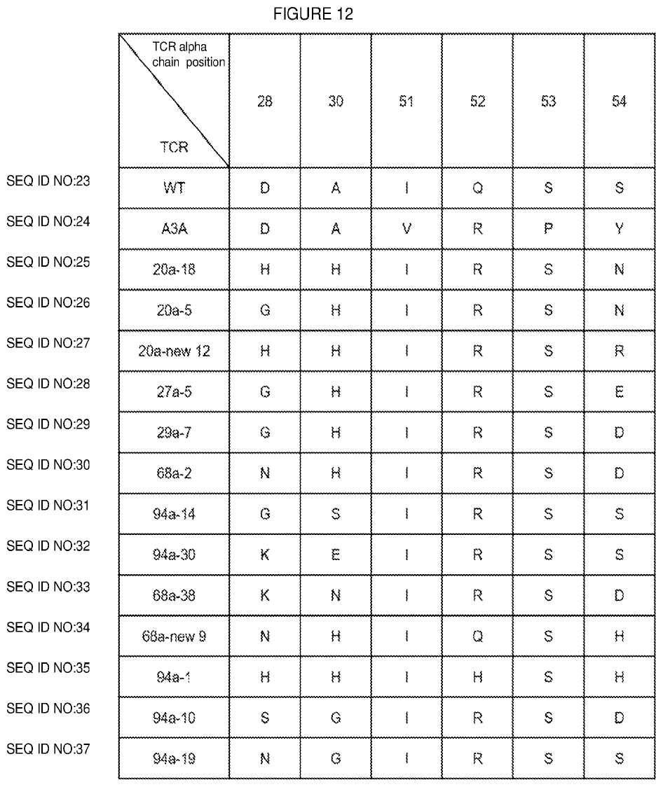

FIG. 11 shows identification of several MAGE TCR mutants with high potency but lower affinity compared to A3A TCR. A. Correlation between Emax and HLA-A1-MAGEA3 tetramer stained-positive percentage of WT TCR, A3A TCR, 8 high-potency mutants and 5 intermediate-potency mutants. B. Correlation between Emax and 3D affinity (3D K_{D}) of immobilized HLA-A1-MAGEA3 binding to WT, A3A or 6 other selected TCR mutants. C. Correlation between EC50 and 3D K_{D} of immobilized HLA-A1-MAGEA3 binding to WT, A3A or 6 other selected TCR mutants.

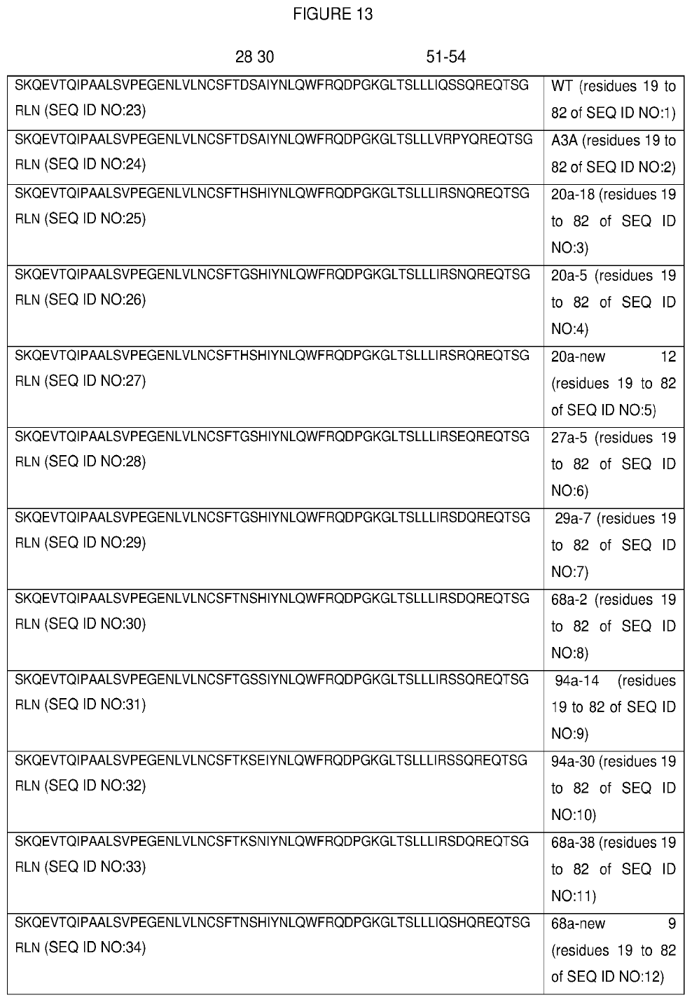

FIG. 13. The protein sequence of MAGEA3 WT TCR alpha chain (SEQ ID NO:1) and beta chain (SEQ ID NO:16). All the mutants only have mutations in TCR alpha chain. A. MAGEA3 WT TCR alpha chain protein sequence. The highlight and underlined residues in TCR alpha chain are Asp28, Ala30, Ile51, Gln52, Ser53 and Ser54. MAGEA3 WT TCR beta chain protein sequence.

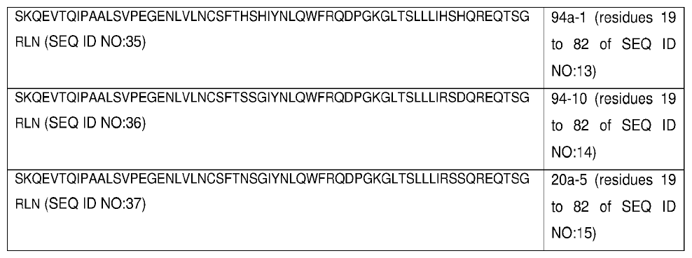

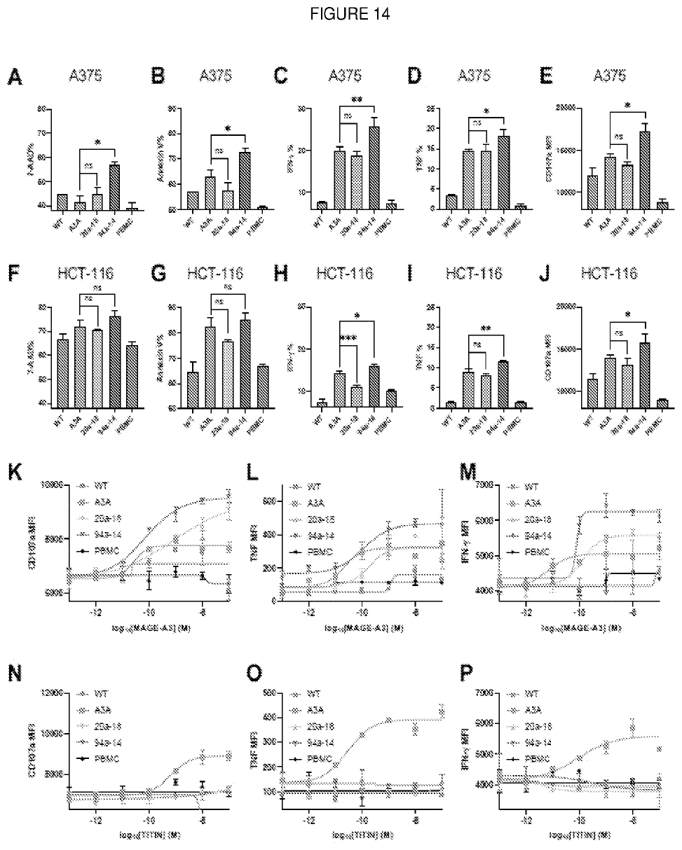

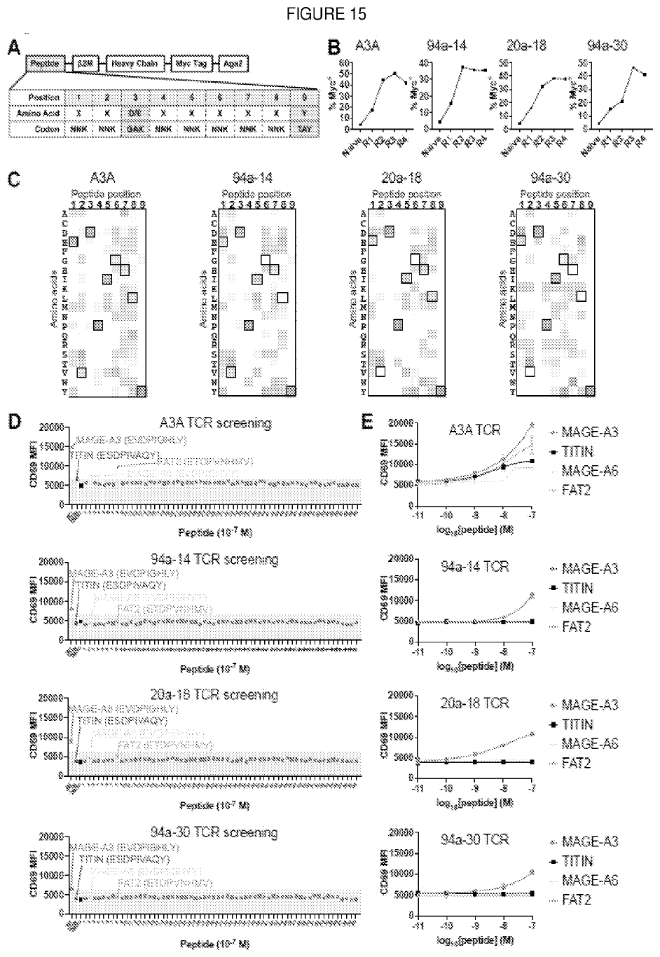

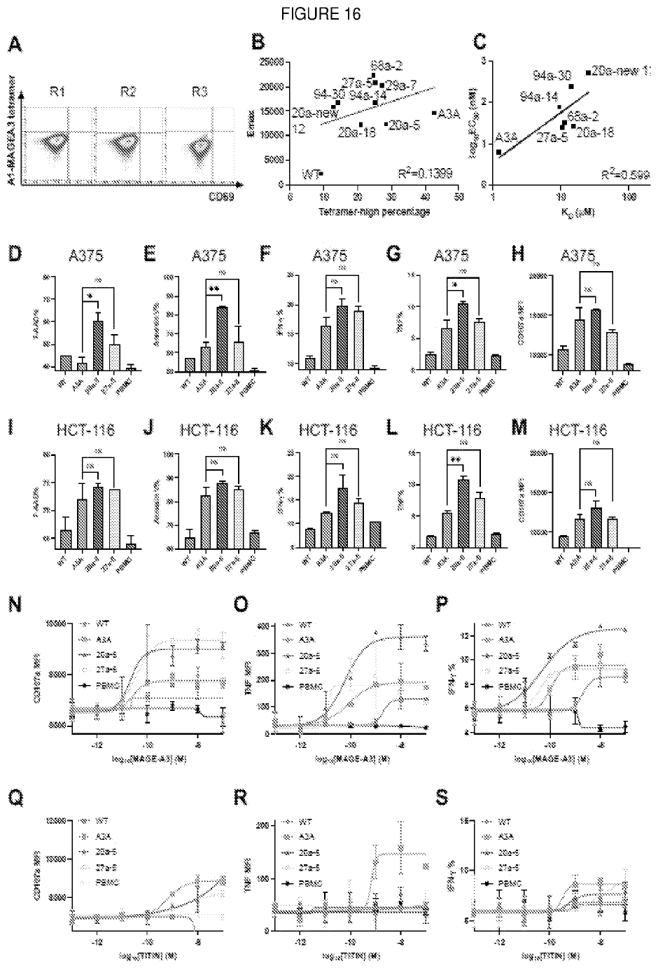

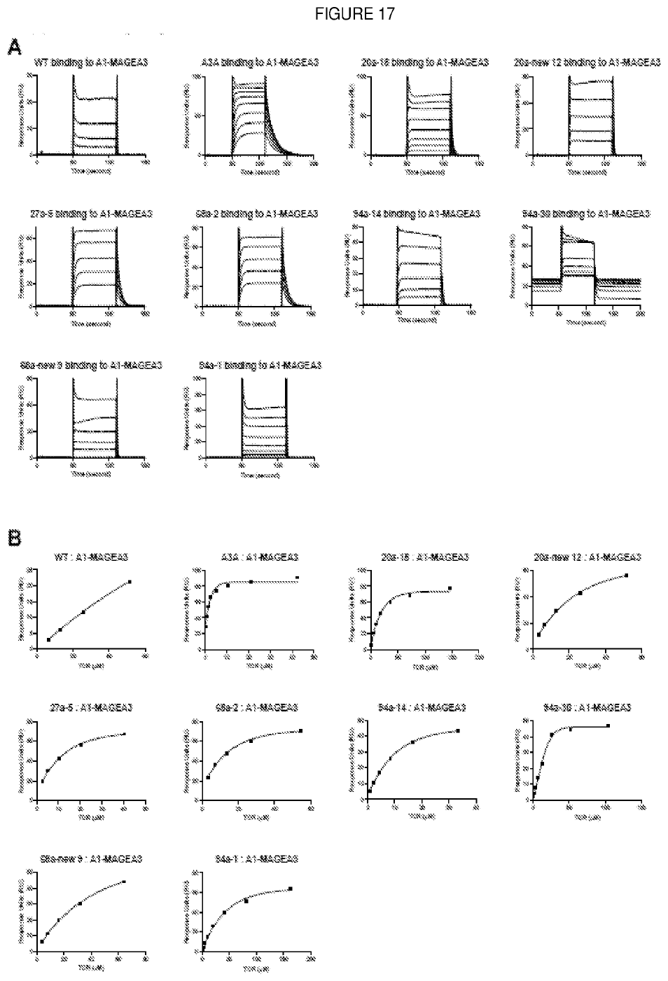

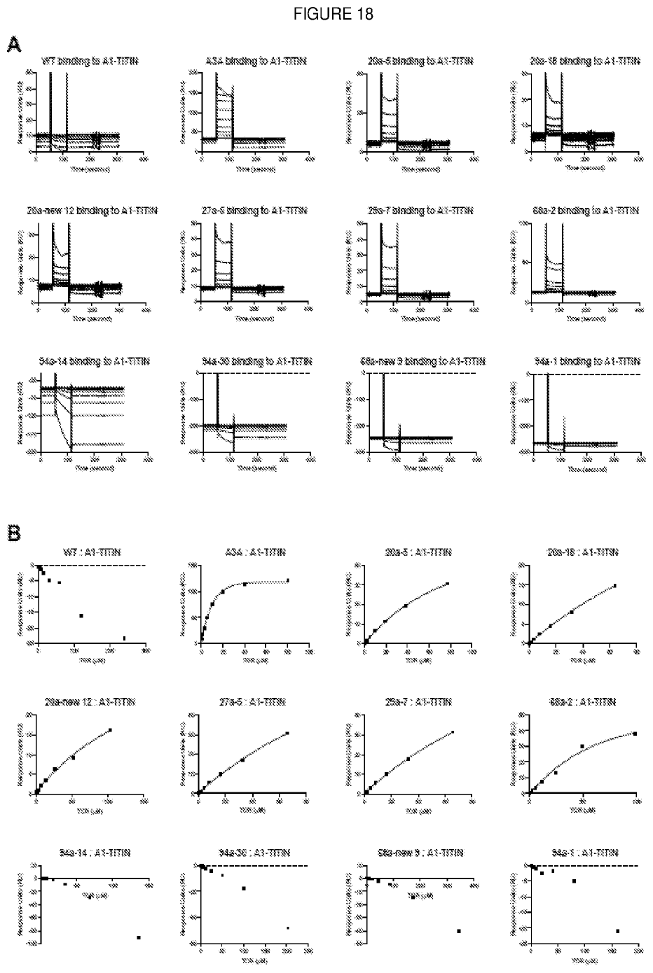

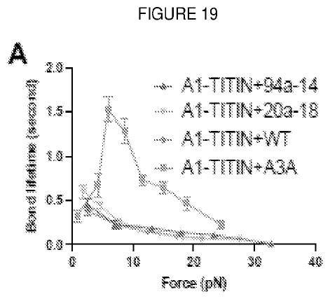

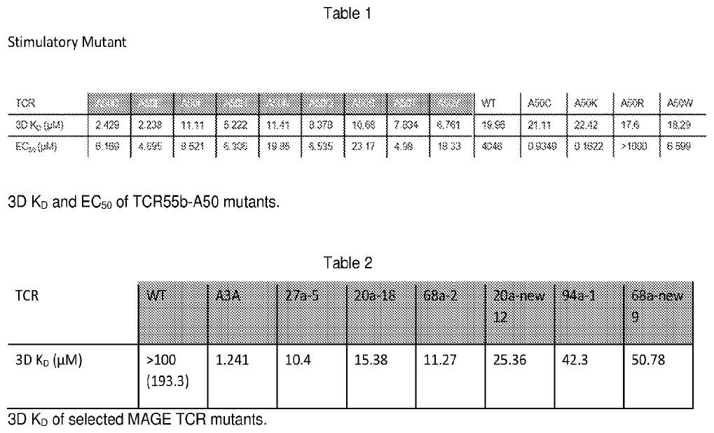

Shown in FIG. 14 are sequences of all MAGE TCR mutants, SEQ ID NO:1-16. All the mutants only have mutations in TCR alpha chain residues of Asp28, Ala30, Ile51, Gln52, Ser53 and Ser54. The specific mutated residues of each mutate are listed here.

### Materials and Methods

Cell Lines. Cell lines were kept in a humidified incubator at 37° C. with 5% CO₂ unless otherwise denoted. Primary human T cells were cultured in RPMI (ThermoFisher), 10% heat inactivated FCS, 2% heat inactivated human AB serum, 100 U/ml penicillin G, 100 ug/ml streptomycin, 2 mM glutamine. IL-2 (Peprotech) was added to a final concentration of 100 U/ml. Work done with blood samples was conducted in accordance with the rules and regulations of the Stanford institutional review board.

T cell lines were cultured in RPMI+glutamax (Invitrogen) supplemented with 10% FBS supplemented with 5 mM HEPES pH 8.0 (ThermoFisher), and 50 U/ml penicillin and streptomycin (ThermoFisher). KG-1 cells are HLA-B35*01 expressing cells derived from a male with acute myelogenous leukemia. KG-1 cells were used as antigen presenting cells and were cultured in IMDM (ThermoFisher)+10% FBS and 50 U/ml penicillin and streptomycin (ThermoFisher).

Tetramer enrichment and T cell cloning. Tetramer enriched cells were single cell sorted into a round bottom 96-well plate containing 100 μl media (RPMI, 10% heat inactivated FCS, 2% heat inactivated human AB serum, 100 U/ml penicillin G, 100 ug/ml streptomycin, 2 mM glutamine) with a BD Aria cell sorter. Feeder cells were prepared from PBMCs from 2-3 random HLA buffy coats irradiated with 4000 rads in a cesium-137 irradiator. JY cells (Sigma-Aldrich) were irradiated with 12000 rads. 100 μl containing 75,000 PBMCs, 7,500 JY cells, and 160,000 anti-CD3/anti-CD28 beads (Dynal/Invitrogen) were added to each well after sorting. IL-2 (Peprotech) was added to a final concentration of 100 U/ml. Cells were kept in a humidified incubator at 37° C. with 5% CO₂. IL-2 and media were changed as needed.

Lentiviral transduction of TCRs. TCR a and 3 chains were cloned separately into lentiviral vectors. Plasmid DNA sequence integrity were verified by automated fluorescent dideoxy (Sanger) sequencing (Sequetech). 1×10⁶ Phoenix (293) cells were plated in 3.5 mls of DMEM complete media (10% FBS, 10 mM HEPES, Pen-strep, L-glutamate) in a 6-well plate. In a cryo-vial (Fisher). 182 μL of unsupplemented DMEM (Thermo Fisher) was mixed with 18 μL of FuGENE (Promega) was incubated at room temperature for 5 minutes. 5.5 μg of DNA from either TCRα, TCRβ, or CD3 vectors was mixed with 1.1 μg of pCL-10A (Novus Biolgicals) and added to DMEM-FuGENE mixture and left to incubate for 30 minutes at room temperature. Transfection mixtures for TCRα, TCRβ, or CD3 encoding plasmids were added to separate wells of Phoenix cells and left overnight at 37° C. Media was changed the following day and transferred to a 32° C. incubator. The next morning, supernatants were harvested and collected, and replaced with fresh complete DMEM. Supernatant was kept at 4° C. The next day supernatants were harvested, collected and combined (TCRa, TCRb, and CD3). Supernatants containing virus were filtered through an 0.45 μm filter and concentrated using a 100 kDa spin filter (Amicon) until the total volume reached 0.5-1 ml. Concentrated virus was buffer exchanged into RPMI complete using 100 kDA filter and again concentrated to 0.5-1 ml. Concentrated virus was added to 2×10⁶ either cells in 12 well plates. Plates containing cells and virus were spun at 2500 rpm for 2 hr at 32° C. After centrifugation plates were returned to 37° C. incubator. TCR expression was checked by antibody and tetramer staining via flow cytometry after 5 days. TCR⁺ CD3⁺ population was sorted for further use.

CD69 upregulation. T cells were rested overnight or for 2-3 hours in fresh RPMI complete. KG-1 antigen presenting cells were pulsed with desired concentration of peptide for 2-3 hours incubated at 37° C. KG-1 cells were washed to remove excess peptide and re-suspended with rested SKW3 T cells. Cells were co-cultured for 14 hours. Cells were stained with anti-CD3 (UCHT-1, BD Biosciences) (1:100) and anti-hCD69 (1:100) (Biolegend) for 1 hour on ice in PBSA (PBS+0.5% BSA). Cells were washed once and analyzed via flow cytometry on an Accuri (BD Biosciences) or Cytoflex (Beckman Coulter). Assay was performed in biological and technical triplicates. EC50 was determined in Prism.

### Example 2

Engineering High-Sensitivity T Cell Receptors with Physiological Affinities Through Catch Bond Recruitment

Adoptive cell therapy using engineered T cell receptors (TCRs) is a promising approach for targeting cancer antigens, but tumor-reactive TCRs are often only weakly responsive to their target ligands (pMHCs). Affinity-matured TCRs can enhance the efficacy of TCR-T therapy but also show target antigen cross-reactivity and recipient organ immunopathology. We developed an alternative strategy, by screening for TCR mutants that paradoxically exhibited high activation signals coupled with low-affinity pMHC binding, through the acquisition of catch bonds, which extend bond lifetimes under shearing forces. Using this approach, we engineered analogs of a clinically-tested tumor antigen MAGE-A3-specific TCR that maintained physiological affinities yet showed equal or greater target killing potency, without acquisition of off-target reactivity, compared to a high-affinity TCR that previously exhibited lethal cross-reactivity with a cardiac antigen. Catch bond recruitment is a biophysically-based strategy to generate potent TCRs for engineered T cell therapy with reduced potential for adverse cross-reactivity.

Here we report an alternative TCR engineering strategy that we call “catch bond fishing” that harnesses a biophysical parameter mediating many adhesive cell surface protein-protein interactions. As a proof of principle experiment, we converted a non-stimulatory TCR into a potently activated TCR by screening of catch bond fishing TCR display libraries that produced TCR with comparable 3D binding affinities to the non-responsive parent TCR clone, but high sensitivity to functional signaling that was in all cases paralleled by the acquisition of catch bonds. We then applied the catch bond engineering strategy to a melanoma antigen MAGE-A3-specific TCR with extremely weak activation and killing capacity. We identified several TCR mutants with target killing potency equal or superior to that of the clinical affinity-matured version of the A3A TCR that showed off target cross-reactivity and toxicity. In spite of this gain in response sensitivity, our catch bond engineered TCRs maintained physiological affinities and did not cross-react with either the known off-target antigen as compared to the affinity-matured clinical TCR or with a wide array of HLA-associated peptides from the human proteome. Together, these findings provide additional strong evidence for the important role of catch bonds in efficacious TCR-ligand interactions and suggest that catch bond recruitment can be exploited as a general approach to overcome a major limitation of TCR-T therapy.

### Results

Design of ‘catch bond fishing’ libraries. Our previous studies showed that although TCR55 binds to an HIV peptide (Pol⁴⁴⁸⁻⁴⁵⁶) presented by the HLA-B35 MHC molecule with physiological affinities, this TCR-pMHC interaction does not produce measurable T cell activation, or form catch bonds during the binding event. However, HIV peptide mutants isolated from HLA-B35 yeast pMHC libraries, such as pep20, gained the capacity to form catch bonds with TCR55 and potently activated T cells bearing this receptor, while maintaining comparable affinity to the non-stimulatory parent pMHC. Here we asked if, in a reciprocal manner, a functional screen could isolate mutants of TCR55 that enable functional T cell responses evoked by the ‘non-stimulatory’ HIV peptide in conjunction with acquisition of catch bond capacity.

While the source of catch bonds in force-dependent triggering has been attributed to multiple structural elements of the TCR, we focused our library design on the TCR-pMHC interface. Our TCR library design was guided by the biophysical characteristics of catch bonds, which are mediated by the transient formation of hydrogen bonds and/or salt bridges encountered during the TCR/pMHC shearing step preceding disengagement. This leads to extended bond lifetimes that manifest as a transient resistive force before unbinding. Thus, our strategy was to lightly mutate the complementarity determining regions (CDR) residues of TCR55 to encode polar or charged amino acids that would act as fishhooks (bait) to probe for H-bonding and/or salt bridging residues (prey) on the pMHC binding surface during disengagement. Importantly, we chose TCR CDR residue positions for the libraries that were too distant from the pMHC to form direct contacts in the bound state, in order to minimize simply selecting for affinity-matured TCRs.

Based on the structure of the TCR55-HIV-B35 complex, three residues on the TCR55α chain and four residues on the TCR55β chain were selected for the library positions (FIG. 1C). The rationale of this library was to select residues in CDR loops of TCR55 within close proximity to the pMHC surface, yet not in direct contact with pMHC. We wished to avoid ‘affinity maturing’ the interaction by randomizing direct TCR/pMHC contacts. Our selected codon usage, VWR, encodes 12 different codons translated as mainly charged and polar residues including glutamine, glutamate, asparagine, aspartate, arginine, lysine, serine, and histidine to increase the chances of forming new polar interactions. Since the practical diversity of a mammalian cell library is approximately 10⁶-10⁷, three randomized residues on TCR55α chain were combined as one library with diversity of 1,728 (Vα library); four randomized residues on TCR55β chain were combined as one library with diversity of 20,736 (Vβ library). Full-length TCR55 libraries were synthesized and cloned into a lentiviral backbone vector. Lentivirus libraries were constructed and used to infect the SKW3 T cell line at low multiplicity of infection, and TCR libraries were expressed on the surface of T cells. The Vα library was paired with the wild type TCR55β chain, and the Vβ library was paired with the wild type TCR55α chain in the transduced SKW3 cells.

We executed functional selections to directly screen for “high-potency/low-affinity” TCR clones that might be indicative of catch bond recruitment, where TCR triggering is decoupled from pMHC binding strength. The libraries were stimulated with 10 μM HIV peptides and sorted for pMHC tetramer-staining low (no higher than the pMHC tetramer staining of WT TCR55) together with co-staining for activation antigen CD69-high (top 5% population based on anti-CD69 MFI) populations to enrich for “low affinity/high potency” TCR mutants.

Single amino acid substitutions in TCR55 trigger activation through catch bond formation. We carried out three rounds of FACS-sorting selections on the TCR55α CDR library (diversity: 1,728) and enriched a population with a tetramer-low, CD69-high staining phenotype. Approximately 100 single cell clones were recovered and individually tested for activation by the HIV(Pol) peptide. The two clones (clone 8 and clone 17) that showed the most potent response to this pMHC ligand (Figure S3C), encoded identical TCR mutations on TCR55α chain—S28G and A98H. To directly examine if the identified mutations conferred increased potency, SKW3 T cells were transduced with the TCR55α-S28G A98H and WT TCR55β chain and stimulated by B35-associated HIV peptide. To deconvolute which mutation was responsible for the activation, we tested the mutations individually (Figure S3D-S3E) and found that the single mutation of alanine to histidine in the TCR55α CDR3 was sufficient to endow the non-responsive TCR55 with the ability to be signal for activation upon exposure to the B35-HIV pMHC.

The 3D affinity of TCR55α-A98H binding to the B35-HIV pMHC was measured by surface plasmon resonance (SPR) as K_{D}=5.9 μM, which is approximately three-fold lower than the WT TCR55 binding to B35-HIV (K_{D}=17 μM) but still in the physiological affinity range for TCR/pMHC interactions, and higher than measured for binding of TCR589 to B35-HIV(K_{D}=4 μM), a receptor-ligand pair with agonist qualities. Biomolecular force probe (BFP) experiments were conducted to determine if TCR55α-A98H forms catch bonds when interacting with B35-HIV. The non-responsive WT TCR55 showed progressively shorter bond lifetime with increasing force, consistent with slip bond formation. In contrast, application of force increased bond lifetime between TCR55α-A98H and B35-HIV, indicating catch bond formation. Analysis of the previously published structure of TCR55 bound to B35-HIV suggests that the residues 065 and T69 on B35 MHC heavy chain molecule might form new bonds with H98 on TCR55α. Q65 or T69 was mutated to alanine, and only the Q65A mutation significantly abrogated the activation of TCR55α-A98H, suggesting the triggering catch bond may involve an interaction between B35-065 and TCR55α-A98H. BFP showed that B35-Q65A-HIV formed catch bonds with TCR55α-A98H but exhibited shorter peak bond lifetimes the B35-HIV/TCR55α-A98H interaction. However, the formation of catch bonds is a dynamic process and alternative residues may also be involved that are not in such close proximity.

Calibrating TCR55 signaling strength by bond lifetime. The acquisition of T cell activation by B35-HIV(Pol) coincident with catch bond formation by a single point mutant of TCR55 provided an opportunity to investigate structure-function relationships between amino acid substitutions and activation strength. We mutated the TCR55α-A98 to 12 different amino acids to investigate how residue identity at this position affected the strength of TCR signaling. We found that in addition to histidine, mutations to aspartate, glutamate, phenylalanine, glutamine, and tyrosine could also enable TCR55 signaling via B35-HIV(Pol) engagement for lymphocyte activation, albeit to different extents. In contrast, mutations to cysteine, lysine, asparagine, arginine, serine, threonine, and tryptophan did not activate TCR55. Therefore, only select polar, aromatic, and charged amino acids replacing residue TCR55α-A98 rendered the mutant able to signal effectively in response to B35-HIV. To investigate if there was a correlation between signaling capacity and binding strength, we measured the 3D affinity via SPR for each of the different TCR55α-A98 mutants binding to B35-HIV pMHC. Most mutants have an affinity in a narrow range between K_{D}=3 μM and K_{D}=20 μM. Surprisingly, neither the maximum CD69 MFI (R²=0.1893) or EC50 (R²=0.02855) of stimulatory mutants were correlated to the SPR affinity of stimulatory mutants, suggesting that 3D affinity could not explain the gain-of-function exhibited by the stimulatory mutants. Indeed, TCR55α-A98W (K_{D}=6.5 μM), a variant that exhibited higher affinity than WT-TCR55 (K_{D}=19 FM), did not enable TCR-dependent activation in response to B35-HIV(Pol). Furthermore, the most ligand-sensitive of the TCR mutants, TCR55α-A98H (K_{D}=5.9 μM), did not have the highest affinity, and the responsive TCR55α-A98Q mutant (K_{D}=8.0 μM) had a lower affinity than the non-responsive TCR55α-A98W mutant (K_{D}=6.5 μM). To investigate if signaling capacity is correlated to the strength of catch bonds, BFP measurements were done for two B35-HIV responsive mutants: TCR55α-A98E and TCR55α-A98Q. We found that Emax was correlated with the peak bond lifetime (R²=0.996), rather than affinity. Thus, the strength of the catch bonds is a key parameter for the discrimination between agonist and non-agonist TCR-pMHC interactions.

We carried out a parallel screen on a TCR55β CDR library (diversity: 20,736) using the same workflow, and identified a TCR55 variant, clone 36, that exhibited a high level of T cell activation by B35-HIV(Pol). Clone 36 contained two mutations: aCDR1 mutation TCR55β-N28Q, and a CDR2 mutation TCR55β-A50D. We identified the isolated TCR55β-A50D mutation as necessary and sufficient to enable T cell activation by B35-HIV. Replacing the TCR553-A50 position with alternative amino acids showed that aspartate, glutamate, phenylalanine, histidine, asparagine, glutamine, serine, threonine, and tyrosine supported TCR55 mutant responses to B35-HIV to different degree, while cysteine, lysine, arginine, and tryptophan did not support effective signaling. The SPR affinities of TCR55 r-A50 mutants were measured and exhibited a range of K_{D}=2-20 μM, similar to the TCR55α mutants, and which fall within the natural physiological range of TCR affinities. There was a better correlation between maximal CD69 MFI versus 3D binding affinity K_{D} (R²=0.7558) among the TCR55β-A50 mutants than the TCR55α-A98 mutants. However, the EC50 was not correlated with the 3D affinity (R²=0.3543), again suggesting that affinity alone was not sufficient to explain the gain of function with these mutants TCR. We therefore again carried out BFP experiments with the TCR55β-A50E, TCR55β-A50D, TCR55β-A50H and TCR55β-A50T mutants. As for the TCR55α mutants, peak bond lifetime correlated with Emax for TCR55β-A50 mutants stimulated by the B35-HIV pMHC ligand (R²=0.8644). Analysis of the crystal structure of TCR55-HIV-B35 complex shows that residues T69 and 072 on the B35-HIV pMHC potentially mediate the formation of new hydrogen bonds with TCR55β-A50E. To test this hypothesis, K562 cells transduced with B35-T69A prevented activation of T cells bearing TCR55β-A50E, whereas B35-Q72A mutation had no effect. BFP measurement showed that B35-T69A-HIV only formed slip bonds with TCR55β-A50E, which also suggests no activation of TCR55β-A50E by B35-T69A-HIV. Thus, this single pairwise interaction is necessary to support catch bond formation and TCR triggering.

Signaling landscape of catch bond engineered TCR. To assess how the catch bond engineered TCR55 mutants affected intracellular signaling in T cells in response to B35-HIV pMHC ligand, we employed a live cell imaging reporter system to measure the activation dynamics of the ERK, p38 and NFAT2 signaling pathways. In these cells, translocation of fluorescent versions of these intracellular signaling molecules can be visualized in real time and quantified on a single cell basis. All three of these reporters have been shown to translocate in response to TCR stimulation independently of CD28 co-stimulation. Upon engagement with HIV peptide-pulsed B35-expressing antigen-presenting cells, reporter Jurkat T cells expressing the indicated catch bond engineered TCR variants displayed enhanced pathway activation when compared to the non-responding parent TCR55, using the stimulatory TCR589 as a positive control. While both TCR55α-A98H and TCR55β-A50E mutants were able to activate the ERK and p38 signaling pathways for a similar duration at the population level, substantial differences in NFAT2 activation dynamics were observed. These results were quantified by single-cell AUC (area under the curve) analysis, which demonstrated significant differences in both ERK and NFAT2 signaling responses for all the tested TCR variants. Due to the substantially lower signal-to-noise ratio of the p38-KTR reporter, we observed more subtle p38 signaling differences that follow the same hierarchy of mean AUC distribution as compared to ERK or NFAT2 activation. We find a strong correlation between mean ERK (R²=0.9370) or NFAT2 (R²=0.9415) AUC distribution and peak bond lifetime, again suggesting that catch bond strength plays a significant role in TCR-ligand engagements that result in functional intracellular signaling.

Applied force activation of TCR at physiological pMHC density. To investigate the triggering of catch bond engineered TCR55 at extremely low but physiologically relevant levels of pMHC (HIV/HLA-B35), we used the recently developed BATTLES technique (Biomechanically-Assisted T-cell Triggering for Large-scale Exogenous-pMHC Screening). BATTLES technique uses temperature-sensitive polymer beads coated with pMHC proteins displayed at physiological densities (3-4.5 pMHCs/cell) to apply ramping forces (estimated maximum magnitude=20-27.5 pN/s) to T cells interacting with bead surfaces. Upon activation of force, we monitored Ca²⁺ signaling (which is correlated with initial T cell triggering) for >1,000 SKW3T cells transduced with engineered TCR55s containing either TCR55α-A98H, TCR55α-A98E, TCR55α-A98Q, TCR55β-A50E, TCR55β-A50H, TCR55β-A50D or TCR55β-A50T substitutions interacting with HIV peptides. While some T cells exhibited sustained increases in cellular Ca²⁺ flux, most cells showed decreasing fluorescence intensities and result in negative accumulated signals, indicating no triggering. This is consistent with prior literature showing that only a small fraction of T cells are activated at low pMHC densities, even with optimal force.

We then examined all engineered TCR55s for events with an integrated per-cell fluorescence over time higher than zero. All tested substitutions except TCR55-A50T yielded higher integrated per-cell Ca²⁺ signals as compared with WT, with the magnitude of the integrated signal showing a strong correlation with measured peak bond lifetimes. These results, using force-induced activation of single T cells, provide evidence that engineered TCRs can drive efficient activation under the low-density pMHC conditions encountered in vivo.

Application of TCR catch bond engineering to TCR-T cell therapy. Experiments with the TCR55 model system show that catch bond engineering can enhance TCR signaling whilst remaining in the physiological affinity regime. This has implications for ACT with TCR-T cells because many wild-type tumor-reactive TCRs have low affinity binding to tumor pMHC and low sensitivity to signaling in response to relevant tumor-associated antigens, resulting in inefficient tumor killing. The melanoma antigen MAGE-A3-specific TCR (WT) was chosen for catch bond engineering. The antigen is HLA-A1-restricted with a reported 3D SPR binding affinity of K_{D}=500 μM to the WT TCR. This TCR shows extremely poor T cell activation in response to the tumor antigen MAGE-A3, while an affinity-matured mutant of the WT MAGE-A3 TCR, A3A TCR, mediates greatly enhanced T cell activation by the same ligand. However, in clinical trials for melanoma the A3A TCR was found to cross-react with presented TITIN peptide, which is expressed mainly in cardiovascular tissue, leading to a high level of cardiotoxicity. Based on the success of promoting T cell activation by introducing new catch bonds in an otherwise non-stimulatory TCR55-ligand pair, we asked whether we could use this approach to improve the sensitivity of the poorly-responsive wild-type TCR to MAGE-A3 ligand while maintaining low affinity to avoid cross-reactivity with TITIN.

We did not have a crystal structure of the low affinity WT TCR complex with HLA-A1-MAGE-A3, but a structure of the affinity-matured version of the TCR with the HLA-A1-MAGE-A3 complex was available. We thus modeled the WT TCR binding to HLA-A1-MAGE-A3 and designed a library on the TCR a chain. Following the design strategy for TCR55, the residues chosen (CDR1a positions 28, 30; CDR2a positions 52, 54) for the library fall within the CDR loops and are relatively close to the pMHC but do not directly contact the pMHC. The SKW3 T cell line was transduced with the library at low MOI and CD69-hi/tetramer-lo clones were selected as described earlier. After three rounds of selection, approximately 100 single cell clones were selected from the enriched population and tested for TCR-dependent activation. We isolated 13 distinct mutant-transduced SKW3 clones that showed enhanced responsiveness to the MAGE-A3 peptide at a concentration unable to trigger T cells expressing the parental WT TCR. By comparing the Emax of the TCR mutants, we defined 8 clones as “high-potency” mutants compared to the A3A TCR, and 5 clones as “intermediate-potency” mutants. We measured SPR 3D K_{D} for six high-potency mutants and two intermediate-potency mutants binding to HLA-A1-MAGE-A3. The affinities ranged from K_{D}=10 to 50 μM, significantly lower affinities than that of A3A which is K_{D}=1.24 μM. We did not observe a correlation between Emax vs 3D affinity (R²=0.3718) but observed a weak correlation between EC50 and affinity (R²=0.5998). We tested if the 8 high-potency mutants showed cross-reactive functional responses to the TITIN peptide. The A3A-transduced SKW3 cells were strongly activated by the TITIN pMHC ligand. Four mutants (20a-18, 20a-new 12, 94a-14, and 94a-30) exhibited no cross-reactivity with the TITIN peptide, whereas the remaining four displayed very weak activation by TITIN only at high peptide concentrations.

We also measured the binding affinity of all catch bond engineered TCR mutants to HLA-A1-TITIN and they had very low or unmeasurable 3D binding affinity (K_{D}>100 μM), whereas A3A affinity for TITIN was K_{D}=7.7 μM. BFP experiments were performed for WT TCR, A3A TCR, TCR mutants 94a-14 and 20a-18, and all formed catch bonds with HLA-A1-MAGE-A3, with the mutant 94a-14 having a higher peak bond lifetime than A3A and WT TCR. The peak bond lifetimes of WT, A3A, 94a-14, and 20a-18 TCR were well correlated to the maximal CD69 MFI measured (R²=0.9781). To date, a force of ˜10 pN for a CD8:TCR:agonist has been demonstrated to promote optimal effector signaling. At ˜10 pN of force 94a-14 TCR has a significantly higher peak bond lifetime than both WT and A3A TCRs. BFP experiments for 94a-14 or 20a-18 TCR with HLA-A1-TITIN indicate that only slip bond formation was observed for both TCRs, consistent with the loss of TITIN cross-reactivity by 94a-14 and 20a-18 TCRs.

To test if the MAGE-A3 TCR mutants could efficiently kill HLA-A1-MAGE-A3+ tumor cells, human primary T cells were transduced with the WT, A3A, and TCR mutants, and cocultured with the HLA-A1-MAGE-A3+ melanoma cell line A375 or HLA-A1-MAGE-A3+ colon cancer cell line HCT-116. In response to A375 cells, the engineered TCRs 94a-14 and 20a-18 were uniformly superior in target killing to the WT TCR and at least comparable, and in some cases superior to A3A in target stimulated effector activity depending on the metric analyzed (IFN-V, TNF, degranulation). In response to HCT-116 cells, which express lower levels of the MAGE-A3 antigen, similar trends were seen. The mutants 20a-5 and 27a-5 were also tested in human primary T cells and showed a high level of cytotoxicity against A375 melanoma cells and HCT-116 colon cancer cells.

To examine if TCR clones 94a-14 and 20a-18 exhibited cross reactivity to TITIN, primary human T cells transduced with the respective TCRs were co-cultured with MAGE-A3 or TITIN peptide-pulsed antigen-presenting cells. While 20a-18 or 94a-14 showed enhanced cytotoxicity, degranulation, and cytokine secretion after coculturing with MAGE-A3 pulsed cells, none of these TCR clones responded to the presented TITIN peptide. Similarly, the 20a-5 and 27a-5 clones mediated potent cytotoxic responses to MAGE-A3 but only minimal cross-reactivity to TITIN at high concentrations of peptide.

Profiling the cross-reactivity of engineered MAGE-A3 TCRs. Although the engineered TCRs lacked substantial reactivity with the TITIN peptide, we asked if the engineered TCRs had acquired new, untoward peptide reactivities as a result of catch bond recruitment. We turned to a previously described yeast-display pMHC library system originally used to characterize the cross-reactivity of TCRs, and to uncover the novel specificities of TCRs derived from tumor-resident T cells. We first generated an HLA-A*01 9-mer peptide library to survey the cross-reactive landscape of the wild-type, affinity matured A3A, and 3 catch bond engineered MAGE-A3 TCR variants. The library was designed based on peptide sequences known to bind HLA-A*01, fixing anchor residues in positions P3 to aspartate and glutamate and P9 to tyrosine to ensure proper presentation of the peptides in the HLA groove. All remaining positions allowed flexibility to all 20 amino acids for a library diversity of 1.8×10⁸.

We performed selections following established methods with soluble, recombinant forms of the wild-type MAGE-A3 (WT) TCR, A3A, 94a-14, 20a-18, or 94a-30. While the WT TCR failed to enrich any yeast clones, presumably due to its very low 3D binding affinity (K_{D}>500 μM) for MAGE-A3, the high affinity A3A and the engineered mutants strongly enriched populations of yeast clones. The selected library pools were subsequently sequenced to isolate individual sequences. We observed that the selected peptides showed strong convergence at the N-terminal end for all the TCR variants, with a lack of C-terminal specificity, as previously described for A3A. Aside from the fixed anchor residues, the P1 GLU, P4 PRO, and P5 ISO showed strong conservation, and notably exist in both MAGE-A3 and TITIN peptides. The three catch-bond engineered TCR variants showed very similar sequence preferences, indicating that the specificities of the TCRs were minimally changed via catch bond engineering.

The deep sequencing data was used to make off-target predictions using previously developed statistical methods. For the A3A TCR, both TITIN and MAGE-A3 were top ranked predictions, ranking as 1 and 7 respectively. However, for the 3 catch bond engineered TCRs, TITIN was not predicted in the top 35 peptides, while the MAGE-A3 peptide was predicted to bind to all 3 catch bond engineered TCRs.

We tested the top 20 putative off-target predictions for the A3A TCR and catch-bond engineered TCRs with T cell activation assays. The top 20 predicted peptides for each TCR were synthesized and used for screening each TCR (60 peptides in total after removing repetitive peptides. For the A3A TCR, we found that, in addition to MAGE-A3 and TITIN, it was also activated by two previously discovered epitopes MAGE-A6 and FAT2. For the 3 catch bond engineered TCRs (94a-14, 20a-18, and 94a-30), only the MAGE-A3 peptide significantly activated the T cells over baseline. For the WT TCR, none of the peptides significantly stimulated the T cells compared to the DMSO control. The collective results of these cross-reactivity profiling experiments shows that the screen could identify both known on- and off-target specificities for the high-affinity A3A TCR, and that catch bond engineering did not introduce untoward specificities corresponding to known sequences in the human proteome. The yeast-display pMHC screen represents a stringent test that shows the absence of unanticipated human antigen cross-reactivity while clearly identifying the source of cardiac toxicity seen with the A3A TCR.

In environments where cell-cell interactions are subject to shear stresses, it has become clear that mechanical force plays an important role in signal transduction by a variety of receptor-ligand systems. Indeed, catch bonds have been observed as a natural signal potentiating mechanism in various low affinity cell surface adhesion systems such as those involving cadherins, selectins, and Notch, and more recently the TCR. Effective TCR signaling upon engagement with an agonist pMHC ligand on an antigen presenting cell involves the formation of catch bonds that extend receptor-ligand interaction lifetime upon application of a pulling force. Indeed, the presence or absence of catch bonding residues in peptide antigens can decouple TCR triggering from conventional measurements of pMHC binding strength. However, the generality of catch bond involvement in effective TCR stimulation by pMHC is unknown and the possible clinical value of this new insight has not been investigated. Here, using a new assay to screen for mutant TCRs with a combination of modest solution affinity but high sensitivity to ligand-induced signaling, we show that TCR with increased catch bond formation dominate among the effective mutant TCRs isolated, and that these newly acquired catch bonds have not predisposed the TCRs to increased human antigen cross-reactivity. This suggests that while slow off rate per se can enable effective TCR signaling upon pMHC binding, catch bonds can play a deterministic role for antigen responsive TCRs.

This biophysical mechanism has clear actionable value in enabling the decoupling of TCR binding strength from TCR signaling efficacy. Indeed, given the ease of identifying such TCR in the screen, our findings suggest that catch bonds may play a substantial role in the overall operational TCR repertoire and help explain the existing discrepancies in the literature between measured solution binding affinities for specific pMHC and the capacity of those pMHC to show agonist properties in terms of T cell activation. This concept makes sense given the motility of T lymphocytes when scanning for ligand on antigen-presenting or target cells. This bulk motion, along with the activity of cellular filipodia, provide tugging or shear forces that would favor prolongation of TCR-ligand interaction by catch bond formation to enable effective phosphatase exclusion as compared to intrinsic slow-off rate binding that could be disrupted by such forces. This finding has direct implications for the emerging field of TCR-T therapy, where the inherently weak “self” tumor reactivity of TCRs presents limitations to clinical activity.

Our selection strategy was critical to successful isolation of ligand-sensitive yet low-affinity clones for several reasons. First, we focused our libraries on polar and charged residues that can maximize the likelihood of mutant substitutions engaging in adventitious polar interactions during TCR/pMHC disengagement. Second, we designed the libraries to focus on residues that were not in direct contact with the pMHC so that the selection did not simply isolate high-affinity (especially slow off-rate) TCRs. We chose residues that were in the “second shell” of TCR CDR residues, i.e., in close proximity to the pMHC surface but too distant to form direct interactions in the ground state complex. These residues would be ideally positioned to act as “hooks” during shearing of the TCR/pMHC interface. Third, our functional selection strategy directly isolated signaling active (CD69-hi), but low affinity (tetramer low) clones. Although the 3D binding affinity K_{D} of the isolated clones does trend to slightly higher affinity than the WT TCRs, the affinities remain firmly in the physiological regime and the 3D binding affinity K_{D} does not correlate with activity, validating the screening principles.

These results show that catch bond engineered TCRs can be “tuned” for sensitivity through scanning different amino acid substitutions at hotspot positions. Such tunability allows for careful curation of clones with the desired balance of activation versus affinity. We emphasize that TCR signaling can be affected by both TCR affinity maturation or catch bond engineering, and they need not to be mutually exclusive. Indeed, there was a weak positive correlation between the TCR mutants' sensitivity and affinity. However, catch bond engineering enables potency enhancement whilst maintaining physiologic affinity, reducing predisposition towards off-target cross-reactivity compared to affinity-matured TCRs.

While the safety of engineered T cell therapy will ultimately depend on the degree of preferential expression of the target tumor antigen versus healthy tissue, the strategy of catch bond engineering to maintain physiological affinity yet strong agonist signaling responses can reduce the chance of unwanted cross-reactivity with other pMHC for clinically-directed TCRs. Enhancing the efficacy of clinical TCRs has generally involved affinity maturation. However, some affinity-matured TCRs have displayed off-target toxicity. The extreme peptide selectivity of catch-bond engineered TCRs may even be helpful in mitigating on-target/off-tumor reactivities by enhancing therapeutic indices based on relative expression levels of the tumor antigen in healthy versus cancerous tissue. Given the relative ease that we isolated such mutants, and the simplicity of the screen, we feel this lends itself well to a general approach in the TCR-T clinical development pipeline. In the case of TCR55, catch bond libraries were designed based on prior knowledge of a solved crystal structure, but in the case of the MAGE-A3 TCR we modeled the structure based on a non-identical but related TCR structure. Thus, we believe that catch bond engineering does not need guidance from cognate crystallographic data. Powerful new web-based artificial intelligence structure prediction tools, and a large database of solved TCR/pMHC complex structures, should enable library design for catch bond engineering to be carried out without specialized structural insights.

### Materials and Methods

Cell lines. SKW3 T cells (DSMZ) were cultured in RPMI-1640+GluMax (Thermo Fisher Scientific) complemented with 10% fetal bovine serum (FBS, Sigma-Aldrich), 10 mM HEPES and 50 U/mL Pen-Strep (Thermo Fisher Scientific) at 37° C. and 5% CO₂. LentiX cells and 293T cells were cultured in DMEM (Thermo Fisher Scientific) supplemented with 10% FBS, 2 mM L-Glutamine, 10 mM HEPES and 50 U/mL Pen-Strep (Thermo Fisher Scientific) at 37° C. and 5% CO₂. KG-1 cells (ATCC) were cultured in IMDM (Thermo Fisher Scientific) supplemented with 10% FBS and 50 U/mL Pen-Strep (Thermo Fisher Scientific) at 37° C. and 5% CO₂. SF9 cells were cultured in SF900-III media (Thermo Fisher) supplemented with 10% FBS and 10 mg/mL gentamicin sulfate (Thermo Fisher) at 27° C. and atmospheric CO₂. Hi5 cells were grown in insect cell culture medium (Expression Systems) supplemented with 10 mg/mL gentamicin sulfate (Thermo Fisher) at 27° C. and atmospheric CO₂. Jurkat cell lines were cultured in RPMI 1640 supplemented with 10% FBS, 2 mM L-Glutamine, 50 U/mL Penicillin, 50 μg/mL Streptomycin, and 50 μM β-mercaptoethanol at 37° C. and 5% CO₂. HEK293T cell line was cultured in DMEM supplemented with 10% FBS, 2 mM L-Glutamine, and 18 mM HEPES at 37° C. and 5% C02.

Packaging of lentivirus. HEK293T-derived LentiX cells were seed in 6-well plate at a density of 3×10⁵ cells/mL (2 mL in total). On the next day, for each well of cells, 750 ng plasmid of interest, 500 ng psPAX, 260 ng pMD2.G were mixed with 4.5 μL Fugene transfection reagent (Promega) in 100 μL Opti-MEM and rested for 20 min. Fresh cRPMI media were added to each well. Then, the DNA/Fugene mixture was added to each well. Optionally, 12 hours after the transfection, the supernatant of each well was replaced with 2 mL fresh cRPMI. 48 hours after the transfection, the supernatant was ready to infect 10⁶ cells.

Cloning of TCR library. The dsDNA of the TCR library was synthesized commercially by GeneArt technology (Thermo Fisher Scientific) and was cloned into pHR lentiviral vector by HiFi assembly (New England Biolabs). Specifically, 20 ng dsDNA of TCR library, 100 ng linearized pHRvector and 10 μL HiFi assembly mastermix were mixed and incubated at 50° C. for 1 hour (do 8 replicates). 10 μL assembly product was analyzed on agarose gel to check the success of assembly. The remaining assembly product was purified by PCR product clean up kit (Qiagen) and eluted in 30 μL water. The electrocompetent cells MegaX DH10B™ T1R Electrocomp™ Cells (Thermo Fisher Scientific) was defrosted on ice for 30 min. Then, 50 μL MegaX cells were mixed with 5 μL (>100 ng) HiFi assembly product. The tube was tapped for three times and incubated on ice for 30 min. The bacteria/DNA mix was then transferred to chilled electroporation cuvette. The electroporation was conducted at 2.0 kV, 200Ω, 25 μF. The cells were immediately recovered in 1000 μL SOC media. The competent cells culture was then recovered at 37° C., 225 rpm for 1 hour. After the recovery, 10 μL and 1000 μL cell culture was plated on the square bioassay dish (Corning) and cultured at 37° C. overnight. The square bioassay dish plated with 10 μL culture was used for calculating the colony forming unit (cfu). All the colonies were scraped from the square bioassay dish and the plasmids were extracted by maxiprep (Qiagen).

TCR library display by T cells. Lentivirus of the TCR library was packaged by the method above. Lentivirus of TCR55 Vα library was titrated and coinfected SKW3 T cells with wild-type TCR55β lentivirus. Lentivirus of TCR55 Vβ library was titrated and coinfected SKW3 T cells with wild-type TCR55α lentivirus. Lentivirus of MAGE library was titrated and coinfected SKW3 T cells with wild-type MAGE-A3 TCR3 lentivirus. 48 hours after the infection, the percentage of TCR-positive population was determined by anti-CD3 (clone OKT3, BioLegend) staining and analyzed by flow cytometry. The titration of lentivirus that led to 20% infection efficiency was used to infect 100-200 million SKW3 T cells to have a low MOI. TCR-positive cells were sorted (Sony SH800S) and used for further sorting selection.

TCR library selection. 10 million KG-1 cells were labelled with CFSE according to manufacturer's protocol (Thermo Fisher Scientific). The KG-1 cells were then pulsed with 10 M HIV peptide for 3 hours at 37° C., 5% CO₂. The KG-1 cells were resuspended at 5×10⁵ cells/mL and aliquoted into 96-well plate at 200 □L per well. The KG-1 cells were washed once to remove excess peptides. The library of 10 million T cells were resuspended at 5×10⁵ cells/mL and aliquoted into the 96-well plate with KG-1 cells at 200 μL per well. After 14-hour activation, the cells were stained with anti-CD69-APC (clone FN50, BioLegend) and B35-HIV-PE tetramer (the method of making pMHC tetramer is described below) on ice for 30 min. Cells were sorted to select tetramer-staining-low (comparable to TCR55 WT T cell's tetramer staining), anti-CD69-staining-high (top 5% in terms of anti-CD69 MFI) population. Cells were sorted into FBS to maintain cell health. Sorted cells were cultured in cRPMI. It took 2 weeks to grow enough cells to continue the next round of selection. After 3-5 rounds of selection, single cell clones were obtained by diluting cells to 2.5 cells/mL and aliquoting 200 μL cell dilution to each well of 96-well U-bottom plate (Corning). It took 2-4 weeks to grow enough number of cells from single cell clone. Each single cell clone was tested by TCR55 signaling assay described below.

Sequencing of TCR mutants. Single cell clones of SKW3 T cells with expected phenotype were used to extract genomic DNA according to the manufacturer's protocol. The TCR mutant DNA fragment was cloned by PCR and ligated into the pHR vector. The product of ligation was used to transform competent E. coli cells and 30 single colonies was picked for sequencing the TCR mutants. More than one TCR sequence might be found in each single cell clone (each T cell might still be transduced with more than one lentiviral particle at the beginning) and each TCR sequence should be tested individually by transducing SKW3 T cells for further TCR activation signaling assay.

TCR55 signaling assay. Peptide was dissolved and titrated in DMSO. KG-1 cells were labelled with CFSE and then resuspended at 5×10⁵ cells/mL. 200 μL KG-1 cells were aliquoted to each well of 96-well U-bottom plate. KG-1 cells were pulsed with titrated peptides for 3 hours at 37° C., 5% CO₂. After that, KG-1 cells were washed once to remove excess peptides. SKW3 T cell transfectants were resuspended at 5×10⁵ cells/mL and 200 μL T cells were added to each well with peptide-pulsed KG-1 cells. The stimulation was performed at 37° C., 5% CO₂ for 14 hours. After the stimulation, the cells were stained with anti-CD69-APC and anti-αβTCR-BV421 (clone IP26, BioLegend) on ice for 30 min and analyzed by CytoFLEX flow cytometer (Beckman). For phosphor-ERK staining, the stimulation was performed for only 15 min at 37° C., 5% C02. After the stimulation, the cells were immediately fixed with 4% PFA and shake for 15 min. The cells were then washed with PBS (0.5% BSA) and permeabilized in ice cold methanol for 30 min on ice. The cells were then washed with PBS (0.5% BSA) for 2 times and stained with 1:50 dilution of anti-pERK1/2 (clone 197G2, Cell Signaling Technology) for 1 hour at room temperature with shaking. The cells were washed once and analyzed by CytoFLEX.

MAGE-A3-specific TCR signaling assay. MAGE-A3 (EVDPIGHLY; SEQ ID NO:19) or TITIN (ESDPIVAQY; SEQ ID NO:20) peptide (80% purity, Elim peptide) was dissolved and titrated in DMSO. HLA-A1-P2A-EGFP lentiviral vector was used to transfect HEK293T cells and GFP-positive cells were sorted and used as antigen-presenting cells (293T-A1). The 293T-A1 cells were resuspended at 5×105 cells/mL and pulsed with titrated peptide for 3 hours at 37° C., 5% CO₂. 200 μL KG-1 cells were aliquoted to each well of 96-well U-bottom plate. After the pulsing, the 293T-A1 cells were washed once to remove excess peptides. MAGE-A3 specific TCR mutants-transduced SKW3 cells were resuspended at 5×10⁵ cells/mL and 200 L T cells were added to each well with peptide-pulsed 293T-A1 cells. The stimulation was performed at 37° C., 5% CO₂ for 14 hours. After the stimulation, the cells were stained with anti-CD69-APC and anti-Vβ5.1-BV421 (clone LC4, ThermoFisher Scientific) on ice for 30 min and analyzed by CytoFLEX flow cytometer (Beckman).

Transduction of human primary T cells with TCR. Human whole blood from healthy anonymous volunteer donors was purchased from Stanford Blook Bank under the approved protocol of APB-2749-KG1018. 6-well plate was coated with 2 mL of 2.5 μg/mL anti-CD3 (OKT3 clone) overnight. The next day, human PBMC were added to the plate with 5 μg/mL anti-CD28 and cultured at 37° C., 5% CO₂ for 3 days. 4 million LentiX cells were seed in 10-cm dish and transfected with lentiviral vector of MAGE-A3-specific TCR a chain or P chain. The lentivirus was made as described above. In total 40 mL of TCR virus were concentrated to 500 μL using 100 kDa-cutoff filter. 5 million preactivated human PBMC were resuspended in 500 μL media and mixed with 500 μL concentrated TCR virus and 5 μg/mL Polybrene and 100 U/mL human IL-2. The virus/cells mixture was processed with spin infection under 2800 rpm, 32° C. for 2 hours.

Killing assay of tumor cells. 20,000 A375 or HCT-116 cells were seed in each well of 96-well plate. 60,000 MAGE-A3-specific TCR-transduced human primary cells were added to each well with tumor cells and cocultured for 24 hours. The plate was washed in EDTA-free buffer and stained with 7-AAD (ThermoFisher Scientific) and Annexin V-APC (BioLegend) for 10 min. The plate was analyzed by CytoFLEX.

Cytotoxicity, cytokine, and granule release assays. 200,000 tumor cells or peptide-pulsed 293T-A1 cells were seeded in each well of 96-well plate overnight. Next day, 200,000 MAGE-A3-specific TCR-transduced human primary cells were mixed with 1:100 anti-CD107a-PE (clone H4A3, BioLegend) and 1:1000 brefeldin A, and then added to each well. Coculture was done for 6 hours at 37° C., 5% CO₂. After 6 hours, the plate was stained with anti-CD8-BV421 (clone RPA-T8, BD Biosciences), anti-Vβ35.1-APC. Then the plate was fixed with IC fixation and permeabilized by permeabilization buffer. The plate was further stained with anti-IFN-γ-BV605 (clone B27, BioLegend) and anti-TNF-PE-Cy7 clone MAbW1, BioLegend) on ice for 30 min. The plate was then washed and analyzed by CytoFLEX.

Production of MHC and β-2-microglobulin inclusion body. The protein of B35 MHC heavy chain and human β-2-microglobulin were made in E. coli as inclusion body. Specifically, B35 MHC heavy chain or human β-2-microglobulin was cloned into pET28a vector and transformed into BL21 (DE3) E. coli strain. Single colony was picked and resuspended in 10 mL LB media containing 50 μg/mL kanamycin and shake at 250 rpm, 37° C. for 12-16 hours. Then the 10 mL culture was added into 1 L LB media containing 50 μg/mL kanamycin and shake at 250 rpm, 37° C. for around 3 hours until the OD=0.5-0.6. IPTG was added into the culture at final concentration of 1 mM and continued to shake for another 3 hours. The bacteria culture was spin down at 6000 rpm for 20 min. The bacteria pellet was resuspended in 50 mL buffer 1 (50 mM Tris-HCl, pH 8.0, 100 mM NaCl, 1 mM DTT, 5% Triton X-100, 1 mM EDTA, 0.2 mM PMSF). Then the bacteria were sonicated under the program of 2 min sonication plus 2 min rest. The sonication program was repeat 4 times continuously. After that, bacteria were spin 7500 rpm for 15 min. It was repeated for two more times to resuspend the bacteria pellet in buffer 1 and do the sonication. The bacteria pellet was then resuspended in 50 mL buffer 2 (50 mM Tris-HCl, pH 8.0, 100 mM NaCl, 1 mM EDTA). Then the bacteria were sonicated under the program of 2 min sonication plus 2 min rest. The sonication program was repeat 4 times continuously. After that, bacteria were spin 7500 rpm for 15 min. It was repeated for one more time to resuspend the bacteria pellet in buffer 2 and do the sonication. The inclusion body was pelleted and solubilized in 25 mL buffer (8 M Urea, 50 mM Tris-HCl pH 8.0, 10 mM EDTA, 10 mM DTT).

Refolding of pMHC. Refolding buffer was prepared as 100 mM Tris-HCl pH 8, 400 mM Arginine, 5 M Urea, 0.5 mM oxidized glutathione, 5 mM reduced glutathione, 2 mM EDTA. 30 mg peptide was dissolved in DMSO and added to each liter of refolding buffer. For each liter of refolding buffer, 30 mg MHC heavy chain inclusion body and 30 mg human β-2-microglobulin inclusion body were mixed in a syringe and added into each liter of refolding buffer drop by drop. Then, the refold buffer/protein were poured into dialysis tubing (Spectrum Labs) and dialyzed into 10 L 10 mM Tris pH 8.0. The 10 L 10 mM Tris pH 8.0 buffer was changed every 12 hours and repeated for 4 times in total. Then, the protein was purified by using weak anion exchange resin (DEAE Cellulose, Santa Cruz Biotechnologies). Specifically, DEAE-Cellulose was equilibrated with 10 mM Tris-HCl, pH 8.0 in a column. Then the dialyzed refolded protein solution flowed through the cellulose column drop by drop and repeated the flowing one more time. The refolded protein was eluted in 30 mL 10 mM Tris-HCl, pH 8.0 plus 0.5 M NaCl. The protein was buffer exchanged into 10 mM Tris-HCl, pH 8.0 and concentrated to 500 μL and biotinylated overnight. Biotinylated refolded protein was analyzed by size exclusion chromatography (Superdex 200, GE Healthcare) and ion exchange (MonoQ, GE Healthcare) on AKTAPurifier (GE Healthcare).

pMHC tetramer For staining each 10 million cells, 20 μg biotinylated pMHC protein and g streptavidin-PE (Thermo Fisher Scientific) were aliquoted. 20% of total amount of streptavidin-PE were added into biotinylated pMHC each time at an interval time of one hour and repeated for 5 times. During the interval time, the tetramer was incubated on ice. The pMHC tetramer was stored at 4° C. overnight before using.

Production of TCR protein by Expi293. The TCR protein used for SPR was produced in Expi293 cells (Thermo Fisher Scientific). Specifically, TCR a chain was cloned into pD649 vector with basic zipper, and TCR β chain was cloned into pD649 vector with acid zipper. 15 μg TCR a chain constructs and 15 ug TCR 3 chain constructs were transfected into 75 million Expi293 cells according to the manufacturer's protocol. 4 days after the transfection, the cell culture was spin down at 400 g for 5 min and the supernatant was saved. 1-fold volume of PBS was added to the supernatant and final concentration of 20 mM Tris-HCl pH 8.0 buffer was added. 2 mL Nickel-NTA was added to the supernatant and the solution was rotated overnight at 4° C. Then, the solution was flowed through a column to collect the Ni-NTA and bound protein. 1×HBS pH 7.2 containing 10 mM Imidazole was used to wash the Ni-NTA and protein once, and the protein was eluted by 1×HBS pH 7.2 containing 300 mM Imidazole. The protein was concentrated in a 30 kDa filter (Millipore) and buffer exchanged in 1×HBS pH 7.2. The protein was purified by size-exclusion chromatography using Superdex200 column on AKTAPurifier (GE Healthcare). The purified protein was collected from the according fraction based on the size and run on SDS-PAGE to check the size and 1:1 stoichiometry.

Production of TCR protein by insect cells. The TCR u chain was cloned into pAcGP67a vector with basic zipper, and TCR β chain was cloned into pAcGP67a vector with acid zipper. 2 μL baculovirus linear DNA and 2 gTCR constructs were mixed with 100 μL Opti-MEM (Thermo Fisher Scientific) and 6.6 μL Fugene (Promega), and rest for 15 min. The mixture was added into 2 million SF9 cells and wait for 6-7 days. The cell culture was spin down at 2000 rpm for 8 min. The supernatant was saved as P0 virus. The P1 virus was made by adding 25 μL P0 virus to 25 mL SF9 cells at 2 million cells/mL. 25 mL media was added to the culture after 24 hours. 6-7 days later, the P1 virus was collected by spinning down the cell culture at 2000 rpm for 8 min and saving the supernatant. The P1 virus of TCR a chain and TCR p chain was used and titrated to coinfect 2 million Hi5 cells to determine the optimal amount of P1 virus used to get the highest amount of 1:1 expression. Usually, 1-4 mL P1 virus for each chain was used for 1 L Hi5 cells (2 million cells/mL). Optimal amount of P1 virus of TCR a chain and TCR β chain was added to Hi5 cells. 72 hours after the coinfection, the cell culture was spin down at 1500 rpm for 15 min. The supernatant was collected, and for each liter of supernatant, 100 mL 1 M Tris pH 8.0, 1 mL 1 M NiCl2, and 1 mL 5 M CaCl₂ was added and stirred for 30 min. After that, the solution was spin down at 6000 rpm for 15 min. The supernatant was collected and 3 mL Ni-NTA was added to each liter of the solution. The solution was stirred for 5 hours or overnight. Then, the solution was filtered through Buchner funnel and the Ni-NTA was transferred to a filter column. The protein-bound Ni-NTA was washed with 500 mL 1×HBS pH 7.2 containing 20 mM Imidazole. Then, the protein was eluted with 15 mL 1×HBS pH 7.2 containing 300 mM Imidazole. The protein was concentrated in a 30 kDa filter and washed once with 1×HBS pH 7.2. The protein was purified by size-exclusion chromatography using Superdex200 column on AKTAPurifier (GE Healthcare). The purified protein was collected from the according fraction based on the size and run on SDS-PAGE to check the size and 1:1 stoichiometry.

Surface plasmon resonance. The affinity of TCR binding to the specific pMHC was measured by surface plasmon resonance on Biacore T100 (GE Healthcare). The refolded pMHC protein was biotinylated and immobilized on streptavidin chip (GE Healthcare). The TCR protein was treated with 3C protease to remove the basic/acid zipper. The pMHC protein was immobilized until a 100-200 RU increase, and the titrated TCR protein was flowed through the flow cell at 25° C. The affinity of steady-state was determined by the Biacore software. No surface regeneration was required because the sample completely returned to the baseline after the dissociation.

BFP assay. The BFP force clamp assay has previously been described in detail. In brief, a T cell of interest were aspirated onto a piezo driven micropipette controlled by Labview (National Instrument) programs. An opposing micropipette as an aspirated RBC biotinylated with EZ-link NHS-PEG-Biotin (Thermo Fisher Scientific). At the apex of this RBC was a streptavidin-maleimide (Sigma-Aldrich) bound glass bead coated with the pMHCs of interest (HLA B35-HIV(Pol448-456), B35-Pep20, A1-MAGE-A3 or A1-TITIN). This RBC:bead complex served as a force probe sensor. Each T cell was repetitively brought into contact, held and then retracted to the distance controlled by the piezo actuator. The retraction and hold phase generated a force on the TCR:MHC bond, which could be altered, based on the distance the T cell was retracted. The position of the edge of the bead was tracked by the high-resolution camera (1,600 frames/sec) with <3 nm displacement precision. The camera then recorded the time it took for the T cell to disengage the glass bead, which can visually be seen by the RBC retracting and the bead returning to its starting position. Multiple repeated cycles (known as force-clamp cycles) could be carried at a single force in order to generate an average bond lifetime between the TCR and peptide:MHC complex. Varying the level of force and recording lifetimes allowed for the determination of the average bond lifetime and the type of bond formation.

Molecular cloning of TCR signaling reporter plasmids. LCAG-HBG and LEG 11-NFAT2 lentiviral expression plasmids were created by Gibson Assembly cloning based on a split-GFP system described previously. EF1α-ERK-KTR-mScarlet or EF1α-p38-KTR-mScarlet lentiviral expression vector was generated by Gibson Assembly cloning based on an ERK-KTR-Clover or a p38-KTR-mCerulean3 plasmid from Markus Covert lab (Addgene #59150 or #59155).

Jurkat ERK and p38-NFAT2 reporter cell lines. To create a live cell nuclear marker with GFP1-10 expression, Jurkat cell line was transduced with the LCAG-HBG lentiviral expression vector. Stable H2B-tBFP+ Jurkat cells were isolated by FACS sorting and transduced with the LE-EKS lentiviral expression vector. Stable ERK-KTR-mScarlet+ Jurkat cells were then isolated by FACS sorting to create the ERK reporter cell line. To create the p38-NFAT2 reporter cell line, H2B-tBFP+ Jurkat cells were transduced with the LE-38KS and the LEG11-NFAT2 lentiviral expression vectors. Stable p38-KTR-mScarlet+ and GFP1-11-NFAT2+ Jurkat cells were isolated by FACS sorting.

Live cell confocal microscopy. Live cell fluorescence time-lapse imaging data were collected using a Leica SP8 microscope with a 63×NA 1.4 oil objective (Biological Imaging Section, Research Technologies Branch, NIAID). Glass-bottom 8-well imaging chambers were coated with poly-D-lysine overnight at 4° C. and washed twice with PBS. Cells were imaged in a heated 37° C. environment with 5% CO₂. Imaging data were processed by Imaris Cell module, customized Batch analysis, and TranslocQ pipelines.

BATTLES. To produce thermo-responsive ‘smart beads’ (˜47 μm in diameter), we generated a mixture of N-Isopropylacrylamide (NIAPM, 9.2% w/v), poly(ethylene glycol) diacrylate (PEGDA, MW=700, 2.8437% v/v), lanthanide nanophosphors, sodium acrylate (1M, 5.5% v/v) and lithium phenyl-2,4,6-trimethylbenzoylphosphinate (LAP, 39.2 mg/mL, 2.5% v/v). We then injected this mixture and a fluorinated HFE7500 oil suspension with 2% ionic Krytox 157 FSH surfactant and 0.05% v/v acrylic acid into a microfluidic droplet generator to produce water-in-oil droplets that were subsequently polymerized into solid beads under flood UV light (IntelliRay, UV0338) at 100% amplitude (7″ away from the lamp, power=˜50-60 mW/cm2) for 2 minutes (48). After polymerization, carboxylated ‘smart beads’ were washed with 2 mL dimethylformamide for 20 s; 2 mL dichloromethane for 10 s; and 2 mL methanol for 20 s prior to being resuspended in 1 mL PBST buffer. To coat ‘smart beads’ with streptavidin, we preactivated ˜200,000 beads with 1% w/v the N-(3-Dimethylaminopropyl)-N′-ethylcarbodiimide hydrochloride (EDC) in 400 μL 0.1 M MES buffer (pH=4.5) supplemented with 0.01% (v/v) Tween-20 for 3.5 hours at RT on an end-over-end rotator (10 rpm). The beads were spined down, washed with 1 mL 0.1 M borate buffer (pH=8.5) supplemented with 0.01% (v/v) Tween-20 and subsequently resuspended in 400 μL of the same buffer. We then added 16 μL of streptavidin solution (dissolved in 1×PBS at 1 mg/mL) into the mixture and rotated the mixture overnight at 4° C. Next day, we quenched the conjugation reaction by adding 10 μL of 0.25 M ethanolamine in 0.1 M borate buffer (pH=8.5) to the mixture and rotating for 30 minutes at 4° C. The final product was washed 3 times with PBST buffer, resuspended in 200 μL of the same buffer and stored at 4° C. for further use. pMHC functionalized ‘smart beads’ were generated by mixing 0.5 μL of 10 nM biotin-pMHCs with ˜20,000 streptavidin ‘smart’ beads in 50 μL PBST buffer. A PDMS microwell array (1440 wells) was then used to colocalized the pMHC coated beads and the calcium dye (Cal-250, 2 μM) stained T cells. To exert mechanical load on bead-associated T cells, the chip was heated to and maintained at 37° C. for 1 min and then cooled to and kept at 34° C. for 2 min. Immediately after cooling, we acquired a total of 150 Ca2+ fluorescence images at 4 s intervals. Integrated Ca²⁺ signals for single T cells were analyzed by ImageJ and a custom-written MATLAB code.

Yeast-display HLA-A1-peptide library. The yeast-display HLA-A1-peptide library was generated similarly to previously described protocol. To express the HLA-A1-peptide, a single-chain format of peptide library, β-2-microglobulin (β2M) and A1 heavy chain connected by linkers was fused N-terminal to Aga2. The A1 heavy chain contains a Y84A mutation to allow an opening at the terminal of MHC groove and a linker can connect the peptide with p2M. For the peptide library, P3 and P9 were set as anchoring residues with limited diversity: P3 as asparate or glutamate, P9 as tyrosine only. For other positions of peptide library, NNK codon was used to allow all 20 amino acids. The peptide library was synthesized as short nucleotide primers which were amplified via PCR to generate the single chain of pMHC-Aga2 inserts. To generate yeast-display HLA-A1-peptide library, competent EBY-100 yeast cells were electroporated with pMHC-Aga2 library inserts and linear pYAL vector. The pMHC-Aga2 library inserts were ligated to pYAL vector inside yeast cells via homologous recombination. By plating the initial yeast library at 1:10,000, 1:1,000, 1:100, and 1:10, the library size was calculated to have 1.8×10⁸ functional diversity. The yeast library was grown in SDCAA pH 4.5 media. The yeast library was then induced to express the pMHC library protein by growing in SGCAA pH 4.5 media.

Selection of yeast-displayed HLA-A1-peptide library. Yeast-display HLA-A1-peptide library was selected with streptavidin-coated magnetic beads coated with biotinylated TCR proteins. The number of yeast cells used for each round of selection should be 10 times higher than the diversity of the last selection step (Round 1 should use yeast cells number of 10 times of naïve library diversity). The yeast library was first incubated with 250 μL streptavidin magnetic beads in 10 mL PBE buffer (PBS+0.5% FBS+1 mM EDTA) and rotated at 4° C. for 1 hour to do negative selection and remove unspecific binding to streptavidin magnetic beads. After incubation, the yeast-beads mixture was passed through an LS column (Miltenyi) and washed with PBE buffer for 3 times, and all the flow-through was collected. Streptavidin magnetic beads coated with TCR protein was prepared by mixing 400 nM biotinylated TCR monomer with 250 μL streptavidin beads in 4.7 mL PBE buffer for 15 min at 4° C. The flow-through was incubated with TCR-beads for 3 hours at 4° C. on a rotator. The yeast cells were washed and pelleted down at 5000 g for 1 minute. The yeast cells were resuspended in 5 mL PBE buffer and passed through an LS column and washed with PBE buffer for 3 times. The flow-through was discarded. The cells in the column were eluted by 5 mL PBE buffer and pelleted down. The pellet was washed one time with SDCAA media and resuspend again in 3 mL SDCAA media to grow overnight. When the OD is over 2, yeast cells were induced in SGCAA for 2-3 days before the next round of selection. The yeast library was stained with specific TCR tetramer and anti-Myc antibody after each round of selection. The TCR tetramer was prepared at the final concentration of 400 nM by mixing TCR monomer and streptavidin-A647 at the ratio of 5:1. 100,000 yeast cells were stained with TCR tetramer and 2 μL anti-c-Myc-488 antibody (9402S, Cell Signaling) in 200 μL buffer. FACS plots were gated based on the yeast cells induced by SGCAA and stained with streptavidin-A647. Further rounds of selection were repeated with 10×108 yeast with only a modification done to the negative and positive selection using only 50 μL of streptavidin-coated beads with or without TCR in 500 L of PBE.

Deep sequencing. Yeast DNA was extracted by Zymoprep II Kit (Zymo Research) for each round of selection from 50 million yeast cells. Barcoding PCR was firstly done for each DNA sample. The PCR product was purified by gel extraction. The Illumina PCR product was quantified by nanodrop. The amount of each Illumina PCR product and water needed to obtain 40 μL 8 nM solution was calculated, aliquoted and mixed together. We used the Illumina V2 2×300 cycle kit following the manufacturer's protocol for a low diversity library.

Analysis of deep sequencing data and prediction of wild type peptides from yeast selection. The sequencing results were firstly paired by PANDASEQ. The paired sequences were then imported into Geneious software to parse barcodes for each round of selection. Unique peptides were trimmed from the sequences and frequencies of amino acids were counted by custom Perl scripts used prior. To predict wild type peptides for each TCR, a positional frequency matrix was determined based on peptides from round 3 selection. To score 9-mer peptides in the human proteome data, unique peptides counted more than 10 were used to generate position weight matrices (PWM). Each PWM from individual TCR selections were then used to predicted wildtype peptides from human proteome. The Homo sapiens proteome used was from UniProtKB (Proteome ID UP000005640; June 2020 update). Python was used for algorithm for weighted positional frequency matrix and ranking a reference proteome.

Screening of predicted wild type peptides. The top 20 predicted wild type peptides for TCR A3A, 94a-14, 20a-18, 94a-30 were synthesized and there were 59 different peptides all together after removing repetitive peptides. MAGE-A12 was shown to be cross-reactive in a previous study, so the HLA-A1 restricted MAGE-A12 peptide was also synthesized and tested. In total 60 different wild type peptides were used to screen activity of different TCRs. Briefly, 100,000 293-A1 cells were pulsed with different wild type peptides in each well of 96-well plate for 3 hours at 37° C., 5% C02. The 293-A1 cells were then washed with completed RPMI to remove excess peptides. 100,000 SKW3 cells expressing different TCRs were added to each well and cocultured for 14 hours at 37° C., 5% CO₂. Anti-CD69-APC and anti-TCR-BV421 staining of cells were done on ice and analyzed on flow cytometer. To do a dose response of MAGE-A3, TITIN, MAGE-A6, and FAT2 peptides, 100,000 HLA-A1 cells were pulsed with titrated peptides in each well of 96-well plate for 3 hours at 37° C., 5% C02. The 293-A1 cells were then washed one time with completed RPMI to remove excess peptides. 100,000 SKW3 cells expressing different TCRs were added to each well and cocultured for 14 hours at 37° C., 5% CO₂. Anti-CD69-APC and anti-TCR-BV421 staining of cells were done on ice and analyzed on flow cytometer.

All data are expressed as the mean±the standard deviation (SD) (for technical replicates) or mean±the standard error of the mean (SEM) (for biological replicates), is stated in the figure legends, results, and methods details. The exact value of n and what n represents (e.g., number of cells, single molecule ligand binding events or experimental replicates) is stated in figure legends and results. All data except dwell time measurements (in methods details and below) were plotted and analyzed in Prism.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

Sequences

WT (SEQ ID NO: 1, including signal sequence)
METLLGLLILWLQLQWVSSKQEVTQIPAALSVPEGENLVLNCSFTDSAIYNLQWFRQDPGK
GLTSLLLIQSSQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
A3A (SEQ ID NO: 2, including signal sequence)
METLLGLLILWLQLQWVSSKQEVTQIPAALSVPEGENLVLNCSFTDSAIYNLQWFRQDPGK
GLTSLLLVRPYQREQTSGRLNASLDKSSGRSTLYIAASQPGDSATYLCAVRPGGAGSYQLT
FGKGTKLSVIPNIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEKSFETDTNLNF
QNLSVIGFRILLLKVAGFNLLMTLRLWSS
20a-18 (SEQ ID NO: 3, including signal sequence)
METLLGLLILWLQLQWVSSKQEVTQIPAALSVPEGENLVLNCSFTHSHIYNLQWFRQDPGK
GLTSLLLIRSNQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
20a-5 (SEQ ID NO: 4, including signal sequence)
METLLGLLILWLQLQWVSSKQEVTQIPAALSVPEGENLVLNCSFTGSHIYNLQWFRQDPGK
GLTSLLLIRSNQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
20a-new 12 (SEQ ID NO: 5, including signal sequence)
METLLGLLILWLQLQWVSSKQEVTQIPAALSVPEGENLVLNCSFTHSHIYNLQWFRQDPGK
GLTSLLLIRSRQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
27a-5 (SEQ ID NO: 6, including signal sequence)
METLLGLLILWLQLQWVSSKQEVTQIPAALSVPEGENLVLNCSFTGSHIYNLQWFRQDPGK
GLTSLLLIRSEQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
29a-7 (SEQ ID NO: 7, including signal sequence)
METLLGLLILWLQLQWVSSKQEVTQIPAALSVPEGENLVLNCSFTGSHIYNLQWFRQDPGK
GLTSLLLIRSDQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
68a-2 (SEQ ID NO: 8, including signal sequence)
METLLGLLILWLQLQWVSSKQEVTQIPAALSVPEGENLVLNCSFTNSHIYNLQWFRQDPGK
GLTSLLLIRSDQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
94a-14 (SEQ ID NO: 9, including signal sequence)
METLLGLLILWLQLQWVSSKQEVTQIPAALSVPEGENLVLNCSFTGSSIYNLQWFRQDPGK
GLTSLLLIRSSQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
94a-30 (SEQ ID NO: 10, including signal sequence)
METLLGLLILWLQLQWVSSKQEVTQIPAALSVPEGENLVLNCSFTKSEIYNLQWFRQDPGK
GLTSLLLIRSSQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
68a-38 (SEQ ID NO: 11, including signal sequence)
METLLGLLILWLQLQWVSSKQEVTQIPAALSVPEGENLVLNCSFTKSNIYNLQWFRQDPGK
GLTSLLLIRSDQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
68a-new 9 (SEQ ID NO: 12, including signal sequence)
METLLGLLILWLQLQWVSSKQEVTQIPAALSVPEGENLVLNCSFTNSHIYNLQWFRQDPGK
GLTSLLLIQSHQREQTSGRLNASLDKSSGRSTLYIAASQPGDSATYLCAVRPGGAGSYQLT
FGKGTKLSVIPNIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEKSFETDTNLNF
QNLSVIGFRILLLKVAGFNLLMTLRLWSS
(SEQ ID NO: 13, including signal sequence)
METLLGLLILWLQLQWVSSKQEVTQIPAALSVPEGENLVLNCSFTHSHIYNLQWFRQDPGK
GLTSLLLIHSHQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS 94a-1 (SEQ ID NO: 13)
94-10 (SEQ ID NO: 14, including signal sequence)
METLLGLLILWLQLQWVSSKQEVTQIPAALSVPEGENLVLNCSFTSSGIYNLQWFRQDPGK
GLTSLLLIRSDQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
20a-5 (SEQ ID NO: 15, including signal sequence)
METLLGLLILWLQLQWVSSKQEVTQIPAALSVPEGENLVLNCSFTNSGIYNLQWFRQDPGK
GLTSLLLIRSSQREQTSGRLNASLDKSSGRSTLYIAASQPGDSATYLCAVRPGGAGSYQLTF
GKGTKLSVIPNIQNPDPAVYQLRDSKSSDKSVCLFTDFDSQTNVSQSKDSDVYITDKTVLDM
RSMDFKSNSAVAWSNKSDFACANAFNNSIIPEDTFFPSPESSCDVKLVEKSFETDTNLNFQ
NLSVIGFRILLLKVAGFNLLMTLRLWSS
SEQ ID NO: 16, including signal sequence
MGSRLLCWVLLCLLGAGPVKAGVTQTPRYLIKTRGQQVTLSCSPISGHRSVSWYQQTPGQ
GLQFLFEYFSETQRNKGNFPGRFSGRQFSNSRSEMNVSTLELGDSALYLCASSPNMADEQ
YFGPGTRLTVTEDLKNVFPPEVAVFEPSEAEISHTQKATLVCLATGFYPDHVELSWWVNGK
EVHSGVSTDPQPLKEQPALNDSRYCLSSRLRVSATFWQNPRNHFRCQVQFYGLSENDEW
TQDRAKPVTQIVSAEAWGRADCGFTSESYQQGVLSATILYEILLGKATLYAVLVSALVLMAM
VKRKDSR
TCR55 alpha chain. The Ala98 hotspot can be mutated to D, E, F, Q,
Y and H to make TCR55 activated by B35-HIV. SEQ ID NO: 17
MLFSSLLCVFVAFSYSGSSVAQKVTQAQSSVSMPVRKAVTLNCLYETSWWSYYIFWYKQL
PSKEMIFLIRQGSDEQNAKSGRYSVNFKKAAKSVALTISALQLEDSAKYFCALGEGGAQKLV
FGQGTRLTINPNIQNPDPAVYQLRDSKSSDKSVCLFTDFDSQTNVSQSKDSDVYITDKTVLD
MRSMDFKSNSAVAWSNKSDFACANAFNNSIIPEDTFFPSPESSCDVKLVEKSFETDTNLNF
QNLSVIGFRILLLKVAGFNLLMTLRLWSS
TCR55 beta chain. The Ala50 hotspot can be mutated to D, E, F, H, N,
Q, S, T and Y to make TCR55 activated by B35-HIV. SEQ ID NO: 18
MSIGLLCCVAFSLLWASPVNAGVTQTPKFQVLKTGQSMTLQCAQDMNHNSMYWYRQDPG
MGLRLIYYSASEGTTDKGEVPNGYNVSRLNKREFSLRLESAAPSQTSVYFCASRTRGGTLI
EQYFGPGTRLTVTEDLKNVFPPEVAVFEPSEAEISHTQKATLVCLATGFYPDHVELSWWVN
GKEVHSGVSTDPQPLKEQPALNDSRYCLSSRLRVSATFWQNPRNHFRCQVQFYGLSEND
EWTQDRAKPVTQIVSAEAWGRADCGFTSESYQQGVLSATILYEILLGKATLYAVLVSALVLM
AMVKRKDSR

1. An engineered αβ T cell receptor (TCR) specific for human MAGE-A3, comprising an alpha chain of SEQ ID NO:1, or a mature version thereof, that comprises at least one amino acid modification to enhance target activation potency, at one or more residues selected from D28, A30, I51, Q52, S53 and S54, wherein the numbering is made relative to the mature polypeptide sequence.

2. The engineered αβ TCR of claim 1, wherein the 3D log K_{D} (μM) for MAGE-A3 and HLA-A1 is from about 0.5 to about 100 μM.

3. The engineered αβ TCR of claim 1, wherein the TCR p chain comprises the sequence of SEQ ID NO:16 or a mature version thereof.

4. The engineered αβ TCR of claim 1, wherein the amino acid modification is one or more of D28H, D28N, D28, D28K, D28S; A30H, A30S, A30E, A30N, A30G; I51V; Q52R, Q52H; S53P; S54Y, S54N, S54R, S54E, S54D, S54H.

5. The engineered αβ TCR of claim 1, wherein the TCRα has a sequence selected from SEQ ID NO:2-SEQ ID NO:15, or a variant derived therefrom.

6. The engineered αβ TCR of claim 1, wherein the TCRα has a sequence with at least 95% sequence identity to a sequence selected from SEQ ID NO:2-SEQ ID NO:15.

7. An engineered αβ T cell receptor (TCR) specific for human HLA-B35 and an HIV peptide, comprising an alpha chain of SEQ ID NO:17 and a beta chain of SEQ ID NO:18, comprising at least one amino acid modifications selected from SEQ ID NO:17 A98D, A98E, A98F, A98Q, A98Y, A98H and SEQ ID NO:18 A50D, A50E, A50F, A50H, A50N, A50Q, A50S, A50T, A50Y.

8. A polynucleotide encoding a TCR alpha or beta chain according to claim 1.

9. A vector comprising a polynucleotide of claim 8.

10. The vector of claim 9, wherein the TCR sequence is operably linked to a promoter.

11. The vector of claim 9, wherein the vector is integrated into a host cell genome.

12. An engineered cell comprising a polynucleotide of claim 8.

13. A therapeutically effective dose of an engineered cell population comprising a polynucleotide of claim 8.

14. The engineered cell or cell population of claim 12, wherein the polynucleotide is integrated into the genome of the cell.

15. The engineered cell or cell population of claim 12, wherein the cell is a T cell.

16. The engineered cell or cell population of claim 12, wherein the cell is selected from naïve CD8⁺ T cells, cytotoxic CD8⁺ T cells, naïve CD4⁺ T cells, helper T cells, e.g. T_{H}1, T_{H}2, T_{H}9, T_{H}11, T_{H}22, T_{FH}; regulatory T cells, e.g. T_{R}1, natural T_{Reg}, inducible T_{Reg}; memory T cells, e.g. central memory T cells, effector memory T cells, NKT cells, γδ T cells.

17. A method of treating an individual for a MAGE-A3 expressing cancer, the method comprising administering to the individual an effective dose of an engineered cell population according to claim 12, wherein there is increased killing of the cancer cells by the engineered cells.

18. The method of claim 17, wherein the treatment is combined with an additional cancer therapy.

19. The method of claim 17, wherein the cancer is a melanoma.

20. The method of claim 17, wherein the cancer is selected from melanoma, small cell lung cancer, hematologic malignancies, neoplasms of breast, skin, glioma, neuroblastoma, intestine, colorectal, ovary and the kidney.

21. A method of selecting variants of a TCR for target activation potency in combination with reduced off-target cross-reactivity, the method comprising:
generating a library of polynucleotide sequences comprising amino acid variations at pre-determined amino acid residues on CDR loops of a TCR sequence for optimization;
introducing the library into mammalian T cells for expression;
binding to the T cells labeled pMHC multimers of the cognate antigen;
selecting for low levels of binding corresponding to a 3D log K_{D} (μM) of from about 0.1 to about 100 μM;
incubating the pool of selected cells with a cognate antigen source for a period of time sufficient to activate T cells; and
selecting for T cells having high levels of activation.
